(19) United States
(12) Patent Application Publication
(10) Pub. No.: US 2001/0049144 A1
(43) Pub. Date: Dec. 6, 2001

(54) Methods for high level expression of genes in primates

(76) Inventors: Victor Rivera, Arlington, MA (US); Philip Zoltick, Wynnewood, PA (US); James M. Wilson, Gladwyne, PA (US)

Correspondence Address: David L. Berstein, ARIAD Gene Therapeutics, Inc., 26 Landsdowne Street, Cambridge, MA, 02139-4234, US

(21) Appl. No.: 09/733,368

(22) Filed: Dec. 8, 2000

Related U.S. Application Data
(60) Provisional application No. 60/170,019, filed on Dec. 10, 1999.

(51) Int. Cl. A61K 48/00; C12N 15/86

(52) U.S. Cl. 435/456; 424/93.21

(57) ABSTRACT

Methods for high level expression of genes in primates are disclosed. Such methods involve expression of transgenes comprising an RSV promoter and a nucleic acid sequence that is heterologous thereto.

### BACKGROUND

A number of important applications, including for example, gene therapy, production of biological materials and biological research, depend on the ability to introduce into cells, and then express, genes encoding RNAs or proteins of therapeutic, commercial, or experimental value. Various promoters and other regulatory elements have been used to try to achieve the highest level of expression possible. Still, maximizing the level of expression for clinical or other applications in various contexts, including within whole organisms, has remained a challenge.

One promoter that is frequently used and is considered to be the optimal promoter for achieving high levels of expression is the promoter from human cytomegalovirus (CMV). Another promoter that has been modestly successful is the promoter from the avian Rous Sarcoma Virus (RSV). This promoter, which is located in the viral long terminal repeats (LTR), is responsible for transcription of viral RNA but can also act as a reasonably strong promoter of exogenous genes in engineered cells.

In many experiments to date, including direct comparisons in mice and tissue culture cells, the CMV promoter was at least as potent as the RSV promoter, and in most cases, resulted in levels of expression that were many fold higher than with the RSV promoter (see for example: Nathwani et al., (1999) Gene Therapy 6:1456-1468; Zarrin et al., (1999), Biochim Biophys Acta, 1446(1-2):135-139; Lee et al., (1997), Mol Cells 31:495-501; Tong et al., (1999), Hybridoma 18:93-97; DeYoung et al., (1999), Human Gene Therapy 10:1469-1478; Norman et al., (1997), Vaccine 15:801-803.). Such results provided a solid basis for the well-established preference for the hCMV promoter among practitioners of heterologous gene expression. However, in the terribly important case of ectopic gene expression in primates, that preference has now been overturned. We have now discovered, quite unexpectedly, that in comparative studies in primates, the RSV promoter effected levels of expression two orders of magnitude greater than CMV. Our discovery represents an advance in ectopic gene expression in primates of great potential clinical and commercial significance.

### SUMMARY OF THE INVENTION

The present invention addresses a long felt need in gene therapy—higher-level expression of transduced genes, particularly in primates. The invention described herein is a method for genetically engineering a primate for expression of a desired gene, comprising introducing into the primate a transgene comprising an RSV promoter and nucleic acid sequence heterologous to said RSV promoter. The transgene may comprise an RSV promoter operably linked to a nucleic acid comprising a desired ORF, or may comprise an RSV promoter and linked to a primate nucleic acid sequence, for example, one selected to permit insertion of the RSV promoter into the primate genome by homologous recombination. In other words, one embodiment of the invention comprises a method for expressing a transgene in a primate, wherein one or more desired genes operably linked to an RSV promoter is introduced into the primate.

In gene therapy, the choice of vehicle used for delivery is an important parameter for successful expression of the transgene in the target tissue. Currently, viral delivery is the preferred delivery method for gene therapy, however, any method for delivery of the transgene may be used, including injection of naked DNA, liposomes, etc. Thus, in one embodiment, the present invention comprises a method for expressing a transgene in primates, wherein the transgene comprises a desired gene operably linked to an RSV promoter and the vector containing the transgene and regulatory region (i.e. the RSV promoter) is packaged in a virus. The virus may be any virus capable of transducing primate cells, including, but not limited to, adenovirus, AAV, retrovirus, hybrid adeno-AAV, herpesvirus and lentivirus. Since gene therapy is targeted toward correction of physiological defects in humans, the primate is preferably a human. When delivering the transgene, choice of the appropriate target tissue will depend on the particular transgene to be expressed. Thus, the target primate tissue into which the RSV-driven transgene is delivered may be, e.g. liver, muscle, retina, neural tissue, blood, etc. A preferred tissue for expression of the RSV-driven transgene is muscle. The primate cells may be transduced in vivo, or may be transduced ex vivo and then introduced into the animal.

For many applications, it is preferable to have regulatable expression of a target gene. In such applications, introduction of the target gene alone into the primate does not ordinarily result in expression of the protein. Protein expression is triggered in most such cases by addition of a compound which regulates transcription of the target gene. Such regulated expression systems often comprise a set of transcription factors which are constitutively expressed within the cell, but are not transcriptionally active in the absence of the regulating compound. Cells capable of regulated expression contain a first DNA construct (or pair of such constructs) encoding chimeric protein molecules comprising (i) at least one receptor domain capable of binding to a selected ligand and (ii) another protein domain, heterologous with respect to the receptor domain, referred to as the “action” domain. Often the action domain is a transcription activation domain. The chimeric proteins, either alone or in combination with additional chimeric proteins, are capable of triggering the activation of transcription of a target gene. The target gene in these cells is under the transcriptional control of a transcriptional regulatory element responsive to binding of ligand to the ligand binding domain. These cells further contain a target gene whose expression is responsive to the binding of ligand to the ligand binding domain, i.e. to the presence of ligand. Several different regulated expression systems have been described. In the regulated expression system of Schreiber et al (see e.g. U.S. Pat. No. 5,830,462) the chimeric proteins multimerize upon addition of ligand and transcription of the target gene is responsive to the multimerization of the chimeric proteins. Any ligand binding domain may be used in the design of such chimeric proteins for the practice of this invention. Exemplary ligand binding domains include immunophilins, cyclophilins, steroid hormone binding domains and antibiotic binding domains. Examples of such domains are FKBP domains, tetracycline binding domains and progesterone binding domains. In most cases, the target gene preferably encodes a peptide sequence of human origin. Alternative embodiments comprise ligand-mediated systems such as those regulated by tetracycline, RU486 or ecdysone.

In the methods of this invention, the RSV promoter can be used to increase expression of the target genes in a regulated system. In these systems, the genes encoding the chimeric transcriptional regulatory protein(s) is operably linked to an RSV promoter. Using the RSV promoter to drive expression of the regulatory proteins in primates allows them to be expressed at high levels and consequently enables high-level expression of the target gene.

### BRIEF DESCRIPTION OF THE FIGURES

FIG. 1: Map of the vector pZAC2.1-rhEPO, containing the human CMV promoter driving the gene for rhesus erythropoietin.

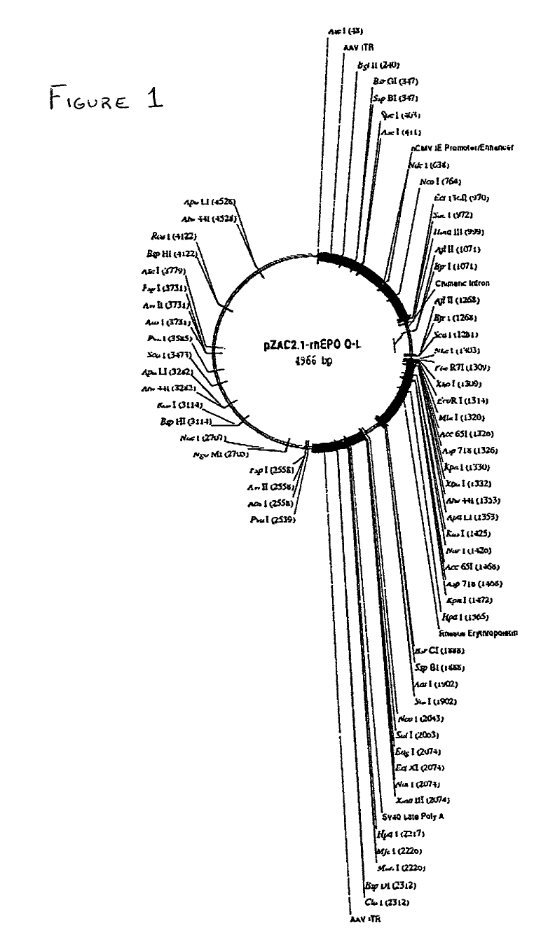

FIG. 2: Map of the vector pZA.RSV-rhEPO, containing the RSV promoter driving the gene for rhesus erythropoietin.

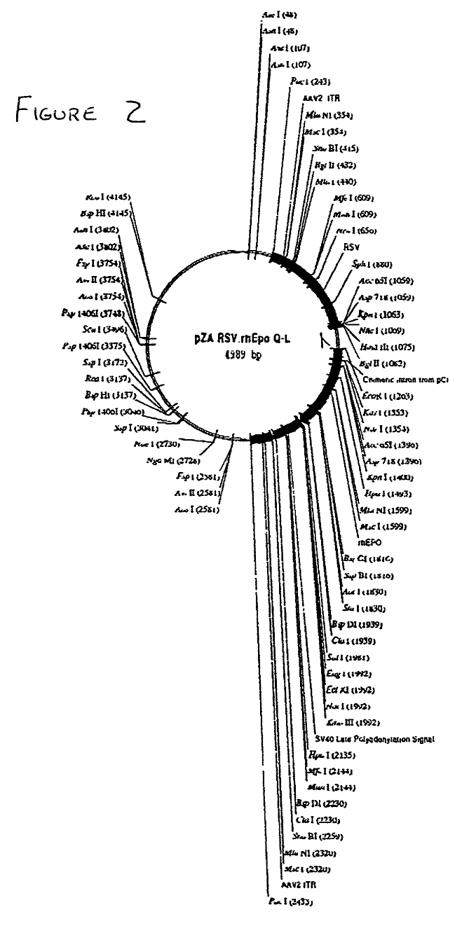

FIG. 3: Comparison between rAAV-CMV-rhEPO and rAAV-RSV-rhEPO in transduced murine muscle.

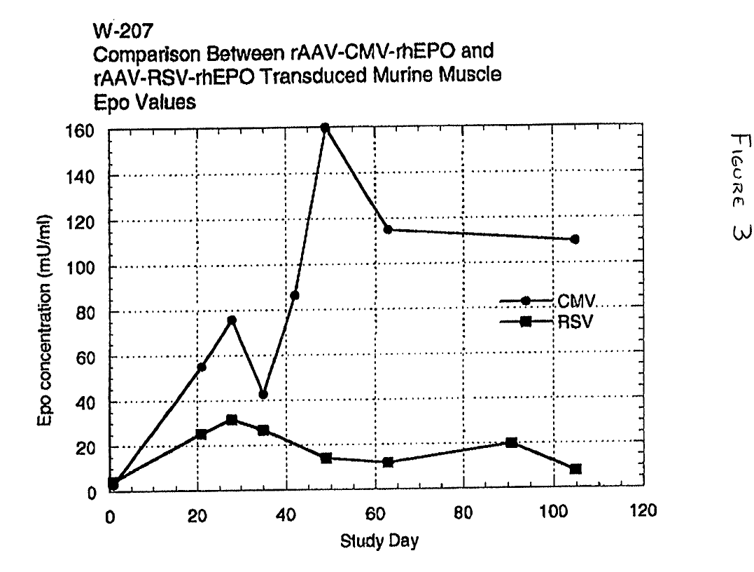

FIG. 4: Serum EPO values following intramuscular transduction with rAAV-CMV-rhEPO.

FIG. 5: Serum EPO values following intramuscular transduction with rAAV-RSV-rhEPO.

### DETAILED DESCRIPTION

Definitions

For convenience, the intended meaning of certain terms and phrases used herein are provided below.

“Activate” as applied to the expression or transcription of a gene denotes a directly or indirectly observable increase in the production of a gene product, e.g., an RNA or polypeptide encoded by the gene.

“Cells”, “host cells” or “recombinant host cells” refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

“Composite”, “fusion”, and “recombinant” denote a material such as a nucleic acid, nucleic acid sequence or polypeptide which contains at least two constituent portions which are mutually heterologous in the sense that they are not otherwise found directly (covalently) linked in nature, e.g. are not found in the same continuous polypeptide or gene in nature, at least not in the same order or orientation or with the same spacing present in the composite, fusion or recombinant product. Such materials contain components derived from at least two different proteins or genes or from at least two non-adjacent portions of the same protein or gene. In general, “composite” refers to portions of different proteins or nucleic acids which are joined together to form a single functional unit, while “fusion” generally refers to two or more functional units which are linked together. “Recombinant” is generally used in the context of nucleic acids or nucleic acid sequences.

A “coding sequence” or a sequence which “encodes” a particular polypeptide or RNA, is a nucleic acid sequence which is transcribed (in the case of DNA) and translated (in the case of mRNA) into a polypeptide in vitro or in vivo when placed under the control of an appropriate expression control sequence. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

The term “conjoint”, with respect to administration of two or more viruses, refers to the simultaneous, sequential or separate dosing of the individual virus provided that some overlap occurs in the simultaneous presence of the viruses in one or more cells of the animal.

A “construct”, e.g., a “nucleic acid construct” or “DNA construct” refers to a nucleic acid or nucleic acid sequence.

“Derived from” indicates a peptide or nucleotide sequence selected from within a given sequence. A peptide or nucleotide sequence derived from a named sequence may contain a small number of modifications relative to the parent sequence, in most cases representing deletion, replacement or insertion of less than about 15%, preferably less than about 10%, and in many cases less than about 5%, of amino acid residues or base pairs present in the parent sequence. In the case of DNAs, one DNA molecule is also considered to be derived from another if the two are capable of selectively hybridizing to one another. Typically, a derived peptide sequence will differ from a parent sequence by the replacement of up to 5 amino acids, in many cases up to 3 amino acids, and very often by 0 or 1 amino acids, Correspondingly, a derived nucleic acid sequence will differ from a parent sequence by the replacement of up to 15 bases, in many cases up to 9 bases, and very often by 0-3 bases. In some cases the amino acid(s) or base(s) is/are deleted rather than replaced.

“Divalent”, as that term is applied to ligands in this document, denotes a ligand which is capable of complexing with at least two protein molecules, e.g., which contain ligand binding domains, to form a three (or greater number)-component complex.

“Domain” refers to a portion of a protein or polypeptide. In the art, “domain” may refer to a discrete 2° structure. However, as will be apparent from the context used herein, the term “domain” is not intended to be limited to a discrete folding domain. Rather, consideration of a polypeptide sequence as a “domain” in, e.g., a fusion protein protein herein, can be made simply by the observation that the polypeptide has a specific activity. Most domains described herein can be derived from proteins ranging from naturally occurring proteins to completely artificial sequences.

“DNA recognition sequence” means a DNA sequence which is capable of binding to one or more DNA-binding domains, e.g., of a transcription factor or an engineered polypeptide.

“Endogenous” refers to molecules which are naturally occurring in a cell, i.e. prior to the genetic engineering or infection of the cell.

“Exogenous” refers to molecules which are not naturally present in the cell, and which have been, e.g., introduced by transfection or transduction of the cell (or the parent cell thereof).

“Gene” refers to a nucleic acid molecule or sequence comprising an open reading frame and including at least one exon and (optionally) an intron sequence. The term “intron” refers to a DNA sequence present in a given gene which is not translated into protein and is generally found between exons.

“Genetically engineered cells” denotes cells which have been modified by the introduction of recombinant or heterologous nucleic acids (e.g. one or more DNA constructs or their RNA counterparts) and further includes the progeny of such cells which retain part or all of such genetic modification.

“Heterologous” as it relates to nucleic acid sequences such as coding sequences and control sequences, denotes sequences that are not normally joined together, and/or are not normally associated with a particular cell. Thus, a “heterologous” region of a nucleic acid construct is a segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, in the case of a cell transduced with a nucleic acid construct which is not normally present in the cell, the cell and the construct would be considered mutually heterologous for purposes of this invention. Allelic variation or naturally occurring mutational events do not give rise to heterologous DNA, as used herein.

“Interact” as used herein is meant to include detectable interactions between molecules, such as can be detected using, for example, a yeast two hybrid assay or by immunoprecipitation. The term interact is also meant to include “binding” interactions between molecules. Interactions may be, for example, protein-protein, protein-nucleic acid, protein-small molecule or small molecule-nucleic acid in nature.

“Ligand” refers to any molecule which is capable of interacting with a corresponding protein or protein domain. A ligand can be naturally occurring, or the ligand can be partially or wholly synthetic. The term “modified ligand” refers to a ligand which has been modified such that it does not significantly interact with the naturally occurring receptor of the ligand in its non modified form.

“Minimal promoter” refers to the minimal expression control sequence that is necessary for initiating transcription of a selected DNA sequence to which it is operably linked.

The terms “promoter” and “expression control sequence” refer to nucleic acid sequences which are associated with transcription of an adjacent ORF, as is well known in the art. Those terms further encompass “tissue specific” promoters and expression control sequences, i.e., promoters and expression control sequences which effect expression of the selected DNA sequence preferentially in specific cells (e.g., cells of a specific tissue). Gene expression occurs preferentially in a specific cell if expression in this cell type is significantly higher than expression in other cell types. The terms “promoter” and “expression control sequence” also encompass so-called “leaky” promoters and “expression control sequences”, which regulate expression of a selected DNA primarily in one tissue, but cause expression in other tissues as well. These terms also encompass non-tissue specific promoters and expression control sequences which are active in most cell types. Furthermore, a promoter or expression control sequence can be constitutive i.e. one which is active basally or inducible, i.e., a promoter or expression control sequence which is active primarily in response to a stimulus. A stimulus can be, e.g., a molecule, such as a hormone, a cytokine, a heavy metal, phorbol esters, cyclic AMP (cAMP), or retinoic acid.

“Nucleic acid” refers to polynucleotides such as deoxyribonucleic acid (DNA), and, where appropriate, ribonucleic acid (RNA), The term should also be understood to include, as equivalents, derivatives, variants and analogs of either RNA or DNA made from nucleotide analogs, and, as applicable to the embodiment being described, single (sense or antisense) and double-stranded polynucleotides.

A “nucleic acid binding domain” refers to a polypeptide which interacts, or binds, with a higher affinity to a nucleic acid having a specific nucleotide sequence relative to a nucleic acid having a nucleotide sequence which is essentially unrelated to the specific nucleotide sequence. In a preferred embodiment, a nucleic acid binding domain is a “DNA binding domain”.

“Oligomerization” and “multimerization”, used interchangeably herein, refer to the association of two or more proteins which can be constitutive or inducible. Constitutive oligomerization refers to direct protein-to-protein association without the need for the mediation of a ligand. Inducible oligomerization is mediated, in the practice of this invention, by the binding of each such protein to a common ligand. “Dimerization” refers to the association of two proteins. The formation of a tripartite (or greater) complex comprising proteins containing one or more FKBP domains together with one or more molecules of an FKBP ligand which is at least divalent (e.g. FK1012 or API 510) is an example of such association or clustering. In cases where at least one of the proteins contains more than one ligand binding domain, e.g., whereat least one of the proteins contains three FKBP domains, the presence of a divalent ligand leads to the clustering of more than two protein molecules. Embodiments in which the ligand is more than divalent (e.g. trivalent) in its ability to bind to proteins bearing ligand binding domains also can result in clustering of more than two protein molecules. The formation of a tripartite complex comprising a protein containing at least one FRB domain, a protein containing at least one FKBP domain and a molecule of rapamycin is another example of such protein clustering. In certain embodiments of this invention, fusion proteins contain multiple FRB and/or FKBP domains. Complexes of such proteins may contain more than one molecule of rapamycin or a derivative thereof and more than one copy of one or more of the constituent proteins. Again, such multimeric complexes are still referred to herein as tripartite complexes to indicate the presence of the three types of constituent molecules, even if one or more are represented by multiple copies. The formation of complexes containing at least one divalent ligand and at least two molecules of a protein which contains at least one ligand binding domain may be referred to as “oligomerization” or “multimerization”, or simply as “dimerization”, “clustering” or association”.

“Operably linked” refers to an arrangement of elements wherein the components so described are configured so as to perform their usual function. Thus, an expression control sequence operably linked to a coding sequence permits expression of the coding sequence. The control sequence need not be contiguous with the coding sequence, so long as it functions to direct the expression thereof. Thus, for example, intervening untranslated yet transcribed sequences can be present between a promoter sequence and the coding sequence and the promoter sequence can still be considered “operably linked” to the coding sequence.

“ORF” or “open reading frame” is a stretch of nucleotides that can be transcribed and translated, resulting in expression of a peptide. The ORF begins at a translation start site (ATG) and ends at a stop codon.

“Protein”, “polypeptide” and “peptide” are used interchangeably herein when referring to a gene product, e.g., as may be encoded by a coding sequence.

A “recombinant virus” is a complete virus particle in which the packaged nucleic acid contains a heterologous portion.

“Subunit”, when referring to the subunit of an activation domain, refers to a portion of the transcription activation domain.

A “target gene” is a nucleic acid of interest, the expression of which is modulated in a regulatable manner by the binding of a ligand to the ligand binding domain of a transgene. The target gene can be endogenous or exogenous and can integrate into a cell's genome, or remain episomal. The target gene can encode a protein or be a non coding nucleic acid, e.g, a nucleic acid which is transcribed into an antisense RNA or a ribozyme.

A “therapeutically effective dose” of a ligand denotes a treatment, e.g., with a dose of ligand which yields detectable alterations in the expression of the target gene.

“Transcription factor” refers to any protein or modified form thereof that is involved in the initiation of transcription but which is not itself a part of the polymerase. Transcription factors are proteins or modified forms thereof, which interact preferentially with specific nucleic acid sequences, i.e., regulatory elements. Some transcription factors are active when they are in the form of a monomer. Alternatively, other transcription factors are active in the form of oligomers consisting of two or more identical proteins or different proteins (heterodimer). The factors have different actions during the transcription initiation: they may interact with other factors, with the RNA polymerase, with the entire complex, with activators, or with DNA. Transcription factors usually contain one or more transcription regulatory domains.

“Transcription regulatory element” is a generic term used throughout the specification to refer to DNA sequences, such as initiation signals, enhancers, and promoters, which induce or control transcription of protein coding sequences with which they are operably linked. The term “enhancer”, also referred to herein as “enhancer element”, is intended to include regulatory elements capable of increasing, stimulating, or enhancing transcription from a minimal promoter. The term “silencer”, also referred to herein as “silencer element” is intended to include regulatory elements capable of decreasing, inhibiting, or repressing transcription from a minimal promoter. Transcription regulatory elements can also be present in genes other than in 5′ flanking sequences. Thus, it is possible that regulatory elements of a gene are located in introns, exons, coding regions, and 3′ flanking sequences.

“Transcription regulatory domain” refers to any domain which regulates transcription, and includes both activation and repression domains. The term “transcription activation domain” denotes a domain in a transcription factor which positively regulates (increases) the rate of gene transcription. The term “transcription repression domain” denotes a domain in a transcription factor which negatively regulates (inhibits or decreases) the rate of gene transcription.

“Transfection” means the introduction of a naked nucleic acid molecule into a recipient cell. “Infection” refers to the process wherein a virus enters the cell in a manner whereby the genetic material of the virus can be expressed in the cell. A “productive infection” refers to the process wherein a virus enters the cell, is replicated, and then released from the cell (sometimes referred to as a “lytic” infection).

“Transduction” encompasses the introduction of nucleic acid into cells by any means.

“Transgene” refers to a nucleic acid sequence which has been introduced into a cell. Daughter cells deriving from a cell in which a transgene has been introduced are also said to contain the transgene (unless it has been deleted). A transgene can encode, e.g., a polypeptide, partly or entirely heterologous to the animal or cell into which it is introduced, or comprises or is derived from an endogenous gene of the animal or cell into which it is introduced, but which is designed to be inserted, or is inserted, into the recipient's genome in such a way as to alter that genome. (e.g., it is inserted at a location which differs from that of the natural gene or under the control of an exogenous transcription control sequence). Alternatively, a transgene can also be present in an episome. A transgene, as used herein, contains an RSV promoter, but can additionally include one or more alternative expression control sequences and any other nucleic acid, (e.g. intron), that may be necessary or desirable for optimal expression of a selected coding sequence. In the context of this invention, the transgene may be a therapeutic gene or it may be a regulatory protein.

“Transient transfection” refers to cases where exogenous DNA does not integrate into the genome of a transfected cell, e.g., where episomal DNA is transcribed into mRNA and translated into protein. A cell has been “stably transfected” with a nucleic acid construct when the nucleic acid construct has been integrated into the genome of that cell.

By “virus” we mean an infective viral particle, comprising a wild type or recombinant nucleic acid genome associated with a capsid protein coat. For example, an adenovirus is a virus particle, comprising an Ad nucleic acid genome associated with an Ad capsid protein coat.

“Wild-type” means naturally occurring in a normal cell or virus.

RSV Promoters

The RSV promoter to be used in conjunction with the methods of this invention is derived from the Long Terminal Repeat (LTR) of a Rous Sarcoma Virus. Numerous commercial cloning vectors are known which contain an RSV promoter sequence. Such cloning vectors include the vector rpDR2 from Clontech and the vector pREP8 from Invitrogen. Alternatively, the RSV promoter may be isolated from any strain of Rous Sarcoma Virus in which the LTR has been shown to have promoter activity. Examples of such strains are shown in the table below:

| RSV Strain | Genbank Accession Number |
|---|---|
| Schmidt-Ruppin A | L29199 |
| Schmidt-Ruppin B | AF052428 |
| Schmidt-Ruppin D | D10652 |
| Prague A | K03367 |
| Prague B | |
| Prague C | J02342 and V01197 |

For example, the RSV promoter used in the Examples comprises the sequence shown below, which is derived from the Schmidt-Ruppin A strain (see Czernilofsky et al., Nucleic Acids Research 8, 2967-2984 (1980)).

| | |
|---|---|
| acgcgtcatgtttgacagcttatcatcgcagatccgtatggtgcactctcagtacaatctgctctgatgccgcatagtta | (Seq. ID #1). |
| agccagtatctgctccctgcttgtgtgttggaggtcgctgagtagtgcgcgagcaaaatttaagctacaacaaggcaagg | |
| cttgaccgacaattgcatgaagaatctgcttagggttaggcgttttgcgctgcttcgcgatgtacgggccagatattcgc | |
| gtatctgaggggactagggtgtgtttaggcgaaaagcggggcttcggttgtacgcggttaggagtcccctcaggatatag | |
| tagtttcgcttttgcatagggagggggaaatgtagtcttatgcaatactcttgtagtcttgcaacatggtaacgatgagt | |
| tagcaacatgccttacaaggagagaaaaagcaccgtgcatgccgattggtggaagtaaggtggtacgatcgtgccttatt | |
| aggaaggcaacagacgggtctgacatggattggacgaaccactaaattccgcattgcagagatattgtatttaagtgcct | |
| agctcgatacaataaacgccatttgaccattcaccacattggtgtgcacctccaagctgggtaccagctgctagcaagct | |
| tgagatct | |

In the above sequence, nucleotides 1-89 correspond to vector sequence, nucleotides 90-125 correspond to the 3′ end of the src coding region, nucleotides 126-348 are unspecified viral sequence, nucleotides 349-612 are from the RSV LTR and nucleotides 613-648 are additional vector sequence. The src coding sequence corresponds to positions 2668-2703 (-488 to -454 relative to the transcription start site) of the Schmidt-Ruppin A Rous Sarcoma Virus env-src-LTR sequence (Genbank Accession number L29199), the unspecified viral sequence corresponds to positions 2704-2926 (−453 to −230) and the LTR region extends from position 2927 to position 3189 (−229 to +31) of Genbank Accession number L29199.

Promoters that are contemplated for use with the methods of this invention include wild type RSV promoter sequences, as well as those with optional changes (including insertions, point mutations or deletions) at certain positions relative to the wild-type promoter. Thus, an RSV promoter of this invention may in some cases vary from naturally occurring RSV promoters by having up to 5 changes per 20 nucleotide stretch. In many embodiments, the natural sequence will be altered in 10 or fewer bases. As used herein, the term “RSV promoter” includes any promoter that can hybridize under stringent conditions of 0.2× SSC, 65° C. to a native RSV promoter, for example, promoters from any strain of RSV listed above. An exemplary promoter is the LTR from the Schmidt-Ruppin A strain, which spans positions 2927-3256 of Genbank Accession number L29199. The RSV promoter may vary in length, comprising from about 50 nucleotides of LTR sequence to 100, 200, 250 or 350 nucleotides of LTR sequence, with or without other viral sequence. Thus, the promoter used may comprise at least 50 nucleotides present in residues 90-612 of seq ID #1, for example, the region spanning positions 550-612 of seq ID #1. Alternatively, the RSV promoter of choice may contain the entire LTR sequence present in Seq ID #1, i.e. positions 349-612, or may have additional viral sequence, such as nucleotides 126-612 or even nucleotides 90-612 of Seq ID #1. As stated above, the term RSV promoter includes any promoter that can hybridize to any of these sequences under stringent conditions.

Transgenes

As used herein, the term “transgene” refers to a nucleic acid sequence that is introduced into a cell. In the context of this invention, the transgene comprises an RSV promoter linked to a heterologous nucleic acid sequence. In one preferred embodiment, the gene is integrated in the chromosomal DNA of a cell. Alternatively, the gene is episomal. A cell comprising a transgene is referred to herein as a “target cell”.

In one embodiment of the invention, the transgene comprises an nucleic acid sequence which is endogenous to the target cell and which is operably linked to RSV promoter. Such a configuration allows the RSV promoter to be inserted by homologous recombination into the genome of the primate cell. In this embodiment, the RSV promoter substitutes for all or a portion of a promoter endogenous to the cell and controls expression of a desired endogenous gene.

In another embodiment, the transgene comprises an RSV promoter linked to a nucleic acid sequence that is heterologous to the target cell. This nucleic acid sequence is preferably an open reading frame encoding a desired protein. In a preferred embodiment, the heterologous gene is integrated into the chromosomal DNA of a cell. The heterologous gene can be inserted into the chromosomal DNA or can substitute for at least a portion of an endogenous gene. The transgene can be present in a single copy or in multiple copies. It is not necessary that the transgene be present in more than one copy. However, if even higher levels of protein encoded by the transgene are desired, multiple copies of the gene can be used.

In still another embodiment, the transgene comprises an RSV promoter linked to a recombinant nucleic acid encoding a chimeric regulatory protein which can be used for activating expression of a target gene in a regulated expression system. In preferred embodiments, a pair of such recombinant nucleic acids is provided, one or both of which are operably linked to an RSV promoter, where the pair of encoded fusion proteins activate transcription of a target gene in a drug-dependent manner.

The transgene in constitutive expression embodiments and the target gene in regulated expression cases comprise a wide variety of genes, including genes that encode a therapeutic protein, antisense sequence or ribozyme of interest. The desired gene can be any sequence of interest which provides a desired phenotype. It can encode a surface membrane protein, a secreted protein, a cytoplasmic protein, or there can be a plurality of genes encoding different products. It may comprise an antisense sequence which can modulate a particular pathway by inhibiting a transcription regulation protein or turn on a particular pathway by inhibiting the translation of an inhibitor of the pathway. It can comprise sequence encoding a ribozyme which may modulate a particular pathway by interfering, at the RNA level, with the expression of a relevant transcription regulator or with the expression of an inhibitor of a particular pathway. The desired proteins which are expressed, singly or in combination, can involve homing, cytotoxicity, proliferation, immune response, inflammatory response, clotting or dissolving of clots, hormonal regulation, etc. The proteins expressed may be naturally-occurring proteins, mutants of naturally-occurring proteins, unique sequences, or combinations thereof.

Various secreted products include hormones, such as insulin, human growth hormone, glucagon, pituitary releasing factor, ACTH, melanotropin, relaxin, etc.; growth factors, such as EGF, IGF-1, TGF-a, TGF-β, PDGF, G-CSF, M-CSF, GM-CSF, FGF, erythropoietin, thrombopoietin, megakaryocytic stimulating and growth factors, etc.; interleukins, such as IL-1 to -13; TNF-a and-b, etc.; receptor antagonists, soluble receptor proteins, etc., enzymes and other factors, such as tissue plasminogen activator, members of the complement cascade, performs, superoxide dismutase, coagulation factors, antithrombin-III, Factor VIIIc, Factor VIIvW, Factor IX, a-antitrypsin, protein C, protein S, endorphins, dynorphin, bone morphogenetic protein, etc. or antibodies.

The gene can encode a naturally-occurring surface membrane protein or a protein made so by introduction of an appropriate signal peptide and transmembrane sequence. Various proteins of interest include homing receptors, e.g. L-selectin (Mel-14), blood-related proteins, particularly having a kringle structure, e.g. Factor VIIIc, Factor VIIIvW, hematopoietic cell markers, e.g. CD3, CD4, CD8, B-cell receptor, TCR subunits a, β, g, d, CD10, CD19, CD28, CD33, CD38, CD41, etc., receptors, such as the interleukin receptors IL-2R, IL-4R, etc., channel proteins for influx or efflux of ions, e.g. Ca+2, K+, Na+, Cl− and the like; CFTR, tyrosine activation motif, ZAP-70, etc.

Proteins may be modified for transport to a vesicle for exocytosis. By adding the sequence from a protein which is directed to vesicles, where the sequence is modified proximal to one or the other terminus, or situated in an analogous position to the protein source, the modified protein will be directed to the Golgi apparatus for packaging in a vesicle. This process in conjunction with the presence of the fusion proteins for exocytosis allows for rapid transfer of the proteins to the extracellular medium and a relatively high localized concentration.

Intracellular and cell surface proteins are also of interest, such as proteins in metabolic pathways, regulatory proteins, steroid receptors, transcription factors, etc., depending upon the nature of the host cell. Some of the proteins indicated above can also serve as intracellular proteins. By way of further illustration, in T-cells, one may wish to introduce genes encoding one or both chains of a T-cell receptor, For B-cells, one could provide the heavy and light chains for an immunoglobulin for secretion. For cutaneous cells, e.g. keratinocytes, particularly stem cell keratinocytes, one could provide for protection against infection, by secreting a-, β-, or g-interferon, antichemotactic factors, proteases specific for bacterial cell wall proteins, etc.

In addition to providing for expression of a gene having therapeutic value, there will be many situations where one may wish to direct a cell to a particular site. The site can include anatomical sites, such as lymph nodes, mucosal tissue, skin, synovium, lung or other internal organs or functional sites, such as clots, injured sites, sites of surgical manipulation, inflammation, infection, etc. By providing for expression of surface membrane proteins which will direct the host cell to the particular site by providing for binding at the host target site to a naturally-occurring epitope, localized concentrations of a secreted product can be achieved. Proteins of interest include homing receptors, e.g. L-selectin, GMP140, CLAM-1, etc., or addressing, e.g. ELAM-1, PNAd, LNAd, etc., clot binding proteins, or cell surface proteins that respond to localized gradients of chemotactic factors. There are numerous situations where one would wish to direct cells to a particular site, where release of a therapeutic product could be of great value.

For use in gene therapy, the desired gene can encode any gene product that is beneficial to a subject. The gene product can be a secreted protein, a membraneous protein, or a cytoplasmic protein. Preferred secreted proteins include growth factors, differentiation factors, cytokines, interleukins, tPA, and erythropoietin. Preferred membraneous proteins include receptors, e.g, growth factor or cytokine receptors or proteins mediating apoptosis, e.g., Fas receptor. Other candidate therapeutic genes are disclosed in U.S. Pat. No. 5,830,462. In yet another embodiment, a “gene activation” construct which, by homologous recombination with genomic DNA, alters the expression control sequences of an endogenous gene, can be used. In such cases, the recombination event (itself under the expression control of an RSV promoter) introduces recognition elements for a DNA binding activity of one a chimeric transcription regulatory protein. A variety of different formats for the gene activation constructs are available. See, for example, the Transkaryotic Therapies, Inc PCT publications WO93/09222, WO95/31560, WO96/2941 1, WO95/31560 and WO94/12650.

Design and assembly of the DNA constructs Constructs may be designed in accordance with the principles, illustrative examples and materials and methods disclosed in the patent documents and scientific literature cited herein, each of which is incorporated herein by reference, with modifications and further exemplification as described herein. Components of the constructs can be prepared in conventional ways, where the coding sequences and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc. as appropriate. In the case of DNA constructs encoding fusion proteins, DNA sequences encoding individual domains and sub-domains are joined such that they constitute a single open reading frame encoding a fusion protein capable of being translated in cells or cell lysates into a single polypeptide harboring all component domains. The DNA construct encoding the fusion protein may then be placed into a vector that directs the expression of the protein in the appropriate cell type(s). For use in the production of proteins in mammalian cells, specifically primate cells, the protein-encoding sequence is introduced into an expression vector that directs expression in these cells under the control of the RSV promoter. Expression vectors suitable for such uses are well known in the art. Various sorts of such vectors are commercially available.

Introduction of Constructs into Cells

This invention is particularly useful for the engineering of primate cells and in applications involving the use of such engineered cells. Human cells are preferred. Across the various primate species, various types of cells may be used, such as hematopoietic, neural, glial, mesenchymal, cutaneous, mucosal, stromal, muscle (including smooth muscle cells), spleen, reticuloendothelial, epithelial, endothelial, hepatic, kidney, gastrointestinal, pulmonary, fibroblast, and other cell types. Of particular interest are muscle cells (including skeletal, cardiac and other muscle cells), hepatic cells, cells of the central and peripheral nervous systems, and hematopoietic cells, which may include any of the nucleated cells which may be involved with the erythroid, lymphoid or myelomonocytic lineages, as well as myoblasts and fibroblasts. Also of interest are stem and progenitor cells, such as hematopoietic, neural, stromal, muscle, hepatic, pulmonary, gastrointestinal and mesenchymal stem cells

The cells may be autologous cells, syngeneic cells, allogeneic cells and even in some cases, xenogeneic cells with respect to an intended host organism. The cells may be modified by changing the major histocompatibility complex (“MHC”) profile, by inactivating β₂-microglobulin to prevent the formation of functional Class I MHC molecules, inactivation of Class II molecules, providing for expression of one or more MHC molecules, enhancing or inactivating cytotoxic capabilities by enhancing or inhibiting the expression of genes associated with the cytotoxic activity, and the like.

In some instances specific clones or oligoclonal cells may be of interest, where the cells have a particular specificity, such as T cells and B cells having a specific antigen specificity or homing target site specificity.

Constructs encoding genes operably linked to the RSV promoter can be introduced into the cells as one or more nucleic acid molecules or constructs, in many cases in association with one or more markers to allow for selection of host cells which contain the construct(s). The constructs can be prepared in conventional ways, where the coding sequences and regulatory regions may be isolated, as appropriate, ligated, cloned in an appropriate cloning host, analyzed by restriction or sequencing, or other convenient means. Particularly, using PCR, individual fragments including all or portions of a functional domain may be isolated, where one or more mutations may be introduced using “primer repair”, ligation, in vitro mutagenesis, etc. as appropriate.

The construct(s) once completed and demonstrated to have the appropriate sequences may then be introduced into a host cell by any convenient means. The constructs may be incorporated into vectors capable of episomal replication (e.g. BPV or EBV vectors) or into vectors designed for integration into the host cells' chromosomes. The constructs may be integrated and packaged into non-replicating, defective viral genomes like Adenovirus, Adeno-associated virus (AAV), Herpes simplex virus (HSV), lentivirus, retrovirus or others, for infection or transduction into cells. Alternatively, the construct may be introduced by protoplast fusion, electroporation, biolistics, calcium phosphate transfection, lipofection, microinjection of DNA or the like. The host cells will in some cases be grown and expanded in culture before introduction of the construct(s), followed by the appropriate treatment for introduction of the construct(s) and integration of the construct(s). The cells may then be expanded and/or screened by virtue of a marker present in the constructs. Various markers which may be used successfully include hprt, neomycin resistance, thymidine kinase, hygromycin resistance, etc., and various cell-surface markers such as Tac, CD8, CD3, Thy1 and the NGF receptor.

In some instances, one may have a target site for homologous recombination, where it is desired that a construct be integrated at a particular locus. For example, one can delete and/or replace an endogenous gene (at the same locus or elsewhere) with a recombinant target construct of this invention. For homologous recombination, one may generally use either Q or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs may be introduced as a single DNA molecule encoding all of the genes, or different DNA molecules having one or more genes. The constructs may be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, and mammalian expression control elements, etc. which may be used to prepare stocks of construct DNAs and for carrying out transfections are well known in the art, and many are commercially available.

Introduction of Constructs into Animals

Any means for the introduction of genetically engineered cells or heterologous DNA into animals, preferably primates, human or non-human, may be adapted to the practice of this invention for the delivery of the various DNA constructs into the intended recipient. by ex vivo genetic engineering Cells which have been transduced ex vivo or in vitro with the DNA constructs may be grown in culture under selective conditions and cells which are selected as having the desired construct(s) may then be expanded and further analyzed, using, for example, the polymerase chain reaction for determining the presence of the construct in the host cells and/or assays for the production of the desired gene product(s). After being transduced with the heterologous genetic constructs, the modified host cells may be identified, selected, grown, characterized, etc. as desired, and then may be used as planned, e.g. grown in culture or introduced into a host organism.

Depending upon the nature of the cells, the cells may be introduced into a host organism, e.g. a mammal, in a wide variety of ways, generally by injection or implantation into the desired tissue or compartment, or a tissue or compartment permitting migration of the cells to their intended destination. Illustrative sites for injection or implantation include the vascular system, bone marrow, muscle, liver, cranium or spinal cord, peritoneum, and skin. Hematopoietic cells, for example, may be administered by injection into the vascular system, there being usually at least about 104 cells and generally not more than about 1010 cells. The number of cells which are employed will depend upon the circumstances, the purpose for the introduction, the lifetime of the cells, the protocol to be used, for example, the number of administrations, the ability of the cells to multiply, the stability of the therapeutic agent, the physiologic need for the therapeutic agent, and the like. Generally, for myoblasts or fibroblasts for example, the number of cells will be at least about 10⁴ and not more than about 10⁹ and may be applied as a dispersion, generally being injected at or near the site of interest. The cells will usually be in a physiologically-acceptable medium.

Cells engineered in accordance with this invention may also be encapsulated, e.g. using conventional biocompatible materials and methods, prior to implantation into the host organism or patient for the production of a therapeutic protein. See e.g. Hguyen et al, Tissue Implant Systems and Methods for Sustaining viable High Cell Densities within a Host, U.S. Pat. No. 5,314,471 (Baxter International, Inc.); Uludag and Sefton, 1993, J Biomed. Mater. Res. 27(10):1213-24 (HepG2 cells/hydroxyethyl methacrylate-methyl methacrylate membranes); Chang et al, 1993, Hum Gene Ther 4(4):433-40 (mouse Ltk-cells expressing hGH/immunoprotective perm-selective alginate microcapsules; Reddy et al, 1993, J Infect Dis 168(4):1082-3 (alginate); Tai and Sun, 1993, FASEB J 7(11):1061-9 (mouse fibroblasts expressing hGH/alginate-poly-L-lysine-alginate membrane); Ao et al, 1995, Transplantation Proc. 27(6):3349, 3350 (alginate); Rajotte et al, 1995, Transplantation Proc. 27(6):3389 (alginate); Lakey et al, 1995, Transplantation Proc. 27(6):3266 (alginate); Korbutt et al, 1995, Transplantation Proc. 27(6):3212 (alginate); Dorian et al, U.S. Pat. No. 5,429,821 (alginate); Emerich et al, 1993, Exp Neurol 122(1):37-47 (polymer-encapsulated PC12 cells); Sagen et al, 1993, J Neurosci 13(6):2415-23 (bovine chromaffin cells encapsulated in semipermeable polymer membrane and implanted into rat spinal subarachnoid space); Aebischer et al, 1994, Exp Neurol 126(2):151-8 (polymer-encapsulated rat PC12 cells implanted into monkeys; see also Aebischer, WO 92/19595); Savelkoul et al, 1994, J Immunol Methods 170(2):185-96 (encapsulated hybridomas producing antibodies; encapsulated transfected cell lines expressing various cytokines); Winn et al, 1 994, PNAS USA 91 (6):2324-8 (engineered BHK cells expressing human nerve growth factor encapsulated in an immunoisolation polymeric device and transplanted into rats); Emerich et al, 1994, Prog Neuropsychopharmacol Biol Psychiatry 18(5):935-46 (polymer-encapsulated PC1 2 cells implanted into rats); Kordower et al, 1994, PNAS USA 91(23):10898-902 (polymer-encapsulated engineered BHK cells expressing hNGF implanted into monkeys) and Butler et al WO 95/04521 (encapsulated device). The cells may then be introduced in encapsulated form into an animal host, preferably a primate and more preferably a human subject in need thereof. Preferably the encapsulating material is semipermeable, permitting release into the host of secreted proteins produced by the encapsulated cells. In many embodiments the semipermeable encapsulation renders the encapsulated cells immunologically isolated from the host organism in which the encapsulated cells are introduced. In those embodiments the cells to be encapsulated may express one or more fusion proteins containing component domains derived from proteins of the host species and/or from viral proteins or proteins from species other than the host species. The cells may be derived from one or more individuals other than the recipient and may be derived from a species other than that of the recipient organism or patient.

by in vivo Genetic Engineering

Instead of ex vivo modification of the cells, in many situations one may wish to modify cells in vivo. A variety of techniques have been developed for genetic engineering of target tissue and cells in vivo, including viral and non-viral systems.

In one approach, the DNA constructs are delivered to cells by transfection, i.e., by delivery to cells of “naked DNA”, lipid-complexed or liposome-formulated DNA, or otherwise formulated DNA. Prior to formulation of DNA, e.g., with lipid, or as in other approaches, prior to incorporation in a final expression vector, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994 (in vivo transfer of an aerosolized recombinant human alphal-antitrypsin gene complexed to cationic liposomes to the lungs of rabbits); Tsan et al, Am J Physiol 268 (Lung Cell Mol Physiol 12): L1052-L1056, 1995 (transfer of genes to rat lungs via tracheal insufflation of plasmid DNA alone or complexed with cationic liposomes); Alton et al., Nat Genet. 5:135-142, 1993 (gene transfer to mouse airways by nebulized delivery of cDNA-liposome complexes). In either case, delivery of vectors or naked or formulated DNA can be carried out by instillation via bronchoscopy, after transfer of viral particles to Ringer's, phosphate buffered saline, or other similar vehicle, or by nebulization.

Viral systems include those based on viruses such as adenovirus, adeno-associated virus, hybrid adeno-AAV, lentivirus and retroviruses, which allow for transduction by infection, and in some cases, integration of the virus or transgene into the host genome. See, for example, Dubensky et al. (1984) Proc. Natl. Acad. Sci. USA 81, 7529-7533; Kaneda et al., (1989) Science 243,375-378; Hiebert et al. (1989) Proc. Natl. Acad. Sci. USA 86, 3594-3598; Hatzoglu et al. (1990) J. Biol. Chem. 265, 17285-17293 and Ferry, et al. (1991) Proc. Natl. Acad. Sci. USA 88, 8377-8381. The virus may be administered by injection (e.g. intravascularly or intramuscularly), inhalation, or other parenteral mode. Non-viral delivery methods such as administration of the DNA via complexes with liposomes or by injection, catheter or biolistics may also be used. See e.g. WO 96/41865, PCT/US97/22454 and WO 99/58700, for example, for additional guidance on formulation and delivery of recombinant nucleic acids to cells and to organisms.

By employing an attenuated or modified retrovirus carrying a target transcriptional initiation region, if desired, one can activate the virus using one of the subject transcription factor constructs, so that the virus may be produced and transduce adjacent cells.

The use of recombinant viruses to deliver the nucleic acid constructs are of particular interest. The transgene(s) may be incorporated into any of a variety of viruses useful in gene therapy.

In clinical settings, the gene delivery systems (i.e., the recombinant nucleic acids in vectors virus, lipid formulation or other form) can be introduced into a patient, e.g., by any of a number of known methods. For instance, a pharmaceutical preparation of the gene delivery system can be introduced systemically, e.g. by intravenous injection, inhalation, etc. In some systems, the means of delivery provides for specific or selective transduction of the construct into desired target cells. This can be achieved by regional or local administration (see U.S. Pat. No. 5,328,470) or by stereotactic injection, e.g. Chen et al., (1994) PNAS USA 91: 3054-3057 or by determinants of the delivery means. For instance, some viral systems have a tissue or cell-type specificity for infection. In some systems cell-type or tissue-type expression is achieved by the use of cell-type or tissue-specific expression control elements controlling expression of the gene.

In preferred embodiments of the invention, the subject expression constructs are derived by incorporation of the genetic construct(s) of interest into viral delivery systems including a recombinant retrovirus, adenovirus, adeno-associated virus (AAV), hybrid adenovirus/AAV, herpes virus or lentivirus (although other applications may be carried out using recombinant bacterial or eukaryotic plasmids). While various viral vectors may be used in the practice of this invention, AAV- and adenovirus-based approaches are of particular interest for the transfer of exogenous genes in vivo, particularly into humans and other primates. The following additional guidance on the choice and use of viral vectors may be helpful to the practitioner, especially with respect to applications involving whole animals (including both human gene therapy and the development and use of animal model systems), whether ex vivo or in vivo.

Viral Vectors:

Adenoviral Vectors

A viral gene delivery system useful in the present invention utilizes adenovirus-derived vectors. Knowledge of the genetic organization of adenovirus, a 36 kb, linear and double-stranded DNA virus, allows substitution of a large piece of adenoviral DNA with foreign sequences up to 8 kb. In contrast to retrovirus, the infection of adenoviral DNA into host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in the human.

Adenovirus is particularly suitable for use as a gene transfer vector because of its mid-sized genome, ease of manipulation, high titer, wide target-cell range, and high infectivity. Both ends of the viral genome contain 100-200 base pair (bp) inverted terminal repeats (ITR), which are cis elements necessary for viral DNA replication and packaging. The early (E) and late (L) regions of the genome contain different transcription domains that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan (1 990) Radiotherap. Oncol. 19:197). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNAs issued from this promoter possess a 5′ tripartite leader (TL) sequence which makes them preferred mRNAs for translation.

The genome of an adenovirus can be manipulated such that it encodes a gene product of interest, but is inactivated in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584). Adenovirus vectors have also been used in vaccine development (Grunhaus and Horwitz (1992) Seminar in Virology 3:237; Graham and Prevec (1992) Biotechnology 20:363). Experiments in administering recombinant adenovirus to different tissues include trachea instillation (Rosenfeld et al. (1991); Rosenfeld et al. (1992) Cell 68:143), muscle injection (Ragot et al. (1993) Nature 361:647), peripheral intravenous injection (Herz and Gerard (1993) Proc. Natl. Acad. Sci. U.S.A. 90:2812), and stereotactic inoculation into the brain (Le Gal La Salle et al. (1993) Science 254:988).

Furthermore, the virus particle is relatively stable and amenable to purification and concentration, and as above, can be modified so as to affect the spectrum of infectivity. Additionally, adenovirus is easy to grow and manipulate and exhibits broad host range in vitro and in vivo. This group of viruses can be obtained in high titers, e.g., 109-1011 plaque-forming unit (PFU)/ml, and they are highly infective. The life cycle of adenovirus does not require integration into the host cell genome. The foreign genes delivered by adenovirus vectors are episomal, and therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors. Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted gene can be under control of, for example, the E1A promoter, the major late promoter (MLP) and associated leader sequences, the viral E3 promoter, or exogenously added promoter sequences such as the RSV promoter.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the method of the present invention. This is because Adenovirus type 5 is a human adenovirus about which a great deal of biochemical and genetic information is known, and it has historically been used for most constructions employing adenovirus as a vector. As stated above, the typical vector according to the present invention is replication defective and will not have an adenovirus E1 region. Thus, it will be most convenient to introduce the nucleic acid of interest at the position from which the E1 coding sequences have been removed. However, the position of insertion of the nucleic acid of interest in a region within the adenovirus sequences is not critical to the present invention. For example, the nucleic acid of interest may also be inserted in lieu of the deleted E3 region in E3 replacement vectors as described previously by Karlsson et. al. (1 986) or in the E4 region where a helper cell line or helper virus complements the E4 defect.

A preferred helper cell line is 293 (ATCC Accession No. CRL1573). This helper cell line, also termed a “packaging cell line” was developed by Frank Graham (Graham et al. (1987) J. Gen. Virol. 36:59-72 and Graham (1977) J. General Virology 68:937-940) and provides E1A and E1B in trans. However, helper cell lines may also be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells.

Various adenovirus vectors have been shown to be of use in the transfer of genes to mammals, including humans. Replication-deficient adenovirus vectors have been used to express marker proteins and CFTR in the pulmonary epithelium. Because of their ability to efficiently infect dividing cells, their tropism for the lung, and the relative ease of generation of high titer stocks, adenoviral vectors have been the subject of much research in the last few years, and various vectors have been used to deliver genes to the lungs of human subjects (Zabner et al., Cell 75:207-216, 1993; Crystal, et al., Nat Genet. 8:42-51, 1994; Boucher, et al., Hum Gene Ther 5:615-639, 1994). The first generation E1 a deleted adenovirus vectors have been improved upon with a second generation that includes a temperature-sensitive E2a viral protein, designed to express less viral protein and thereby make the virally infected cell less of a target for the immune system (Goldman et al., Human Gene Therapy 6:839-851,1995). More recently, a viral vector deleted of all viral open reading frames has been reported (Fisher et al., Virology 217:11-22, 1996). Moreover, it has been shown that expression of viral IL-1 0 inhibits the immune response to adenoviral antigen (Qin et al., Human Gene Therapy 8:1 365-1374, 1997).

Adenoviruses can also be cell type specific, i.e., infect only restricted types of cells and/or express a transgene only in restricted types of cells. For example, the viruses comprise a gene under the transcriptional control of a transcription initiation region specifically regulated by target host cells, as described e.g., in U.S. Pat. No. 5,698,443, by Henderson and Schuur, issued Dec. 16, 1997. Thus, replication competent adenoviruses can be restricted to certain cells by, e.g., inserting a cell specific response element to regulate a synthesis of a protein necessary for replication, e.g., E1A or E1 B.

DNA sequences of a number of adenovirus types are available from Genbank. For example, human adenovirus type 5 has GenBank Accession No.M73260. The adenovirus DNA sequences may be obtained from any of the 42 human adenovirus types currently identified. Various adenovirus strains are available from the American Type Culture Collection, Rockville, Md., or by request from a number of commercial and academic sources. A transgene as described herein may be incorporated into any adenoviral vector and delivery protocol, by the same methods (restriction digest, linker ligation or filling in of ends, and ligation) used to insert the CFTR or other genes into the vectors.

Adenovirus producer cell lines can include one or more of the adenoviral genes E1, E2a, and E4 DNA sequence, for packaging adenovirus vectors in which one or more of these genes have been mutated or deleted are described, e.g., in PCT/US95/15947 (WO 96/18418) by Kadan et al.; PCT/US95/07341 (WO 95/346671) by Kovesdi et al,; PCT/FR94/00624 (WO94/28152) by Imler et al.; PCT/FR94/00851 (WO 95/02697) by Perrocaudet et al., PCT/US95/14793 (WO96/14061) by Wang et al.

AAV Vectors

Another viral vector system useful for delivery of DNA is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129).

AAV has not been associated with the cause of any disease. AAV is not a transforming or oncogenic virus. AAV integration into chromosomes of human cell lines does not cause any significant alteration in the growth properties or morphological characteristics of the cells. These properties of AAV also recommend it as a potentially useful human gene therapy vector.

AAV is also one of the few viruses that may integrate its DNA into non-dividing cells, e.g., pulmonary epithelial cells or muscle cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

The AAV-based expression vector to be used typically includes the 145 nucleotide AAV inverted terminal repeats (ITRs) flanking a restriction site that can be used for subcloning of the transgene, either directly using the restriction site available, or by excision of the transgene with restriction enzymes followed by blunting of the ends, ligation of appropriate DNA linkers, restriction digestion, and ligation into the site between the ITRs. The capacity of AAV vectors is about 4.4 kb. The following proteins have been expressed using various AAV-based vectors, and a variety of promoter/enhancers: neomycin phosphotransferase, chloramphenicol acetyl transferase, Fanconi's anemia gene, cystic fibrosis transmembrane conductance regulator, and granulocyte macrophage colony-stimulating factor (Kotin, R. M., Human Gene Therapy 5:793-801, 1994, Table I). A transgene incorporating the RSV promoter as used in the methods of this invention can similarly be included in an AAV-based vector.

Such a vector can be packaged into AAV virions by reported methods. For example, a human cell line such as 293 can be co-transfected with the AAV-based expression vector and another plasmid containing open reading frames encoding AAV rep and cap (which are obligatory for replication and packaging of the recombinant viral construct) under the control of endogenous AAV promoters or a heterologous promoter. In the absence of helper virus, the rep proteins Rep68 and Rep78 prevent accumulation of the replicative form, but upon superinfection with adenovirus or herpes virus, these proteins permit replication from the ITRs (present only in the construct containing the transgene) and expression of the viral capsid proteins. This system results in packaging of the transgene DNA into AAV virions (Carter, B. J., Current Opinion in Biotechnology 3:533-539, 1992; Kotin, R. M, Human Gene Therapy 5:793-801, 1994)). Typically, three days after transfection, recombinant AAV is harvested from the cells along with adenovirus and the contaminating adenovirus is then inactivated by heat treatment.

Methods to improve the titer of AAV can also be used to express the transgene in an AAV virion. Such strategies include, but are not limited to: stable expression of the ITR-flanked transgene in a cell line followed by transfection with a second plasmid to direct viral packaging; use of a cell line that expresses AAV proteins inducibly, such as temperature-sensitive inducible expression or pharmacologically inducible expression. Alternatively, a cell can be transformed with a first AAV vector including a 5′ ITR, a 3′ ITR flanking a heterologous gene, and a second AAV vector which includes an inducible origin of replication, e.g., SV40 origin of replication, which is capable of being induced by an agent, such as the SV40 T antigen and which includes DNA sequences encoding the AAV rep and cap proteins. Upon induction by an agent, the second AAV vector may replicate to a high copy number, and thereby increased numbers of infectious AAV particles may be generated (see, e.g, U.S. Pat. No. 5,693,531 by Chiorini et al., issued Dec. 2, 1997. In yet another method for producing large amounts of recombinant AAV, a plasmid is used which incorporate the Epstein Barr Nuclear Antigen (EBNA) gene, the latent origin of replication of Epstein Barr virus (oriP) and an AAV genome. These plasmids are maintained as a multicopy extra-chromosomal elements in cells, such as in 293 cells. Upon addition of wild-type helper functions, these cells will produce high amounts of recombinant AAV (U.S. Pat. No. 5,691,176 by Lebkowski et al., issued Nov. 25, 1997). In another system, an AAV packaging plasmid is provided that allows expression of the rep gene, wherein the p5 promoter, which normally controls rep expression, is replaced with a heterologous promoter (U.S. Pat. No. 5,658,776, by Flotte et al., issued Aug.19, 1997). Additionally, one may increase the efficiency of AAV transduction by treating the cells with an agent that facilitates the conversion of the single stranded form to the double stranded form, as described in Wilson et al., WO96/39530.

AAV stocks can be produced as described in Hermonat and Muzyczka (1984) PNAS 81 :6466, modified by using the pAAV/Ad described by Samulski et al. (1989) J. Virol. 63:3822. Concentration and purification of the virus can be achieved by reported methods such as banding in cesium chloride gradients, as was used for the initial report of AAV vector expression in vivo (Flotte, et al. J.Biol. Chem. 268:3781-3790, 1993) or chromatographic purification, as described in O'Riordan et al., WO97/08298.

Methods for in vitro packaging AAV vectors are also available and have the advantage that there is no size limitation of the DNA packaged into the particles (see, U.S. Pat. No. 5,688,676, by Zhou et al., issued Nov. 18, 1997). This procedure involves the preparation of cell free packaging extracts.

For additional detailed guidance on AAV technology which may be useful in the practice of the subject invention, including methods and materials for the incorporation of a transgene, the propagation and purification of the recombinant AAV vector containing the transgene, and its use in transfecting cells and mammals, see e.g. Carter et al, U.S. Pat. No. 4,797,368 (Jan. 10 , 1989); Muzyczka et al, U.S. Pat. No. 5,139,941 (Aug. 18, 1992); Lebkowski et al, US Pat. No. 5,173,414 (Dec. 22, 1992); Srivastava, U.S. Pat. No. 5,252,479 (Oct. 12, 1993); Lebkowski et al, U.S. Pat. No. 5,354,678 (Oct. 11, 1994); Shenk et al, U.S. Pat. No. 5,436,146(Jul. 25, 1995); Chatterjee et al, U.S. Pat. No. 5,454,935 (Dec. 12, 1995), Carter et al WO 93/24641 (published Dec. 9, 1993), and Natsoulis, U.S. Pat. No. 5,622,856 (Apr. 22, 1997). Further information regarding AAVs and the adenovirus or herpes helper functions required can be found in the following articles.Berns and Bohensky (1987), “Adeno-Associated Viruses: An Update”, Advanced in Virus Research, Academic Press, 33:243-306. The genome of AAV is described in Laughlin et al. (1983) “Cloning of infectious adeno-associated virus genomes in bacterial plasmids”, Gene, 23: 65-73. Expression of AAV is described in Beaton et al. (1989) “Expression from the Adeno-associated virus p5 and p19 promoters is negatively regulated in trans by the rep protein”, J. Virol., 63:4450-4454. Construction of rAAV is described in a number of publications: Tratschin et al. (1984) “Adeno-associated virus vector for high frequency integration, expression and rescue of genes in mammalian cells”, Mol. Cell. Biol., 4:2072-2081; Hermonat and Muzyczka (1984) “Use of adeno-associated virus as a mammalian DNA cloning vector: Transduction of neomycin resistance into mammalian tissue culture cells”, Proc. Natl. Acad. Sci. USA, 81:6466-6470; McLaughlin et al. (1988) “Adeno-associated virus general transduction vectors: Analysis of Proviral Structures”, J. Virol., 62:1963-1973; and Samulski et al. (1989) “Helper-free stocks of recombinant adeno-associated viruses: normal integration does require viral gene expression”, J. Virol., 63:3822-3828. Cell lines that can be transformed by rAAV are those described in Lebkowski et al. (1988) “Adeno-associated virus: a vector system for efficient introduction and integration of DNA into a variety of mammalian cell types”, Mol. Cell. Biol., 8:3988-3996. “Producer” or “packaging” cell lines used in manufacturing recombinant retroviruses are described in Dougherty et al. (1989) J. Virol., 63:3209-3212; and Markowitz et al. (1988) J. Virol., 62:1120-1124.

Hybrid Adenovirus-AAV Vectors

Hybrid Adenovirus-AAV vectors represented by an adenovirus capsid containing a nucleic acid comprising a portion of an adenovirus, and 5′ and 3′ ITR sequences from an AAV which flank a selected transgene under the control of a promoter. See e.g. Wilson et al, International Patent Application Publication No. WO 96/1 3598. This hybrid vector is characterized by high titer transgene delivery to a host cell and the ability to stably integrate the transgene into the host cell chromosome in the presence of the rep gene. This virus is capable of infecting virtually all cell types (conferred by its adenovirus sequences) and stable long term transgene integration into the host cell genome (conferred by its AAV sequences).

The adenovirus nucleic acid sequences employed in the this vector can range from a minimum sequence amount, which requires the use of a helper virus to produce the hybrid virus particle, to only selected deletions of adenovirus genes, which deleted gene products can be supplied in the hybrid viral process by a packaging cell. For example, a hybrid virus can comprise the 5′ and 3′ inverted terminal repeat (ITR) sequences of an adenovirus (which function as origins of replication). The left terminal sequence (5′) sequence of the Ad5 genome that can be used spans bp 1 to about 360 of the conventional adenovirus genome (also referred to as map units 0-1) and includes the 5′ ITR and the packaging/enhancer domain. The 3′ adenovirus sequences of the hybrid virus include the right terminal 3′ ITR sequence which is about 580 nucleotides (about bp 35,353-end of the adenovirus, referred to as about map units 98.4-100.

The AAV sequences useful in the hybrid vector are viral sequences from which the rep and cap polypeptide encoding sequences are deleted and are usually the cis acting 5′ and 3′ ITR sequences. Thus, the AAV ITR sequences are flanked by the selected adenovirus sequences and the AAV ITR sequences themselves flank a selected transgene. The preparation of the hybrid vector is further described in detail in published PCT application entitled “Hybrid Adenovirus-AAV Virus and Method of Use Thereof”, WO 96/13598 by Wilson et al.

For additional detailed guidance on adenovirus and hybrid adenovirus-AAV technology which may be useful in the practice of the subject invention, including methods and materials for the incorporation of a transgene, the propagation and purification of recombinant virus containing the transgene, and its use in transfecting cells and mammals, see also Wilson et al, WO 94/28938, WO 96/13597 and WO 96/26285, and references cited therein.

Retroviruses

The retroviruses are a group of single-stranded RNA viruses characterized by an ability to convert their RNA to double-stranded DNA in infected cells by a process of reverse-transcription (Coffin (1990) Retroviridae and their Replication” In Fields, Knipe ed. Virology. New York: Raven Press). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsidal proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and are also required for integration in the host cell genome (Coffin (1990), supra).

In order to construct a retroviral vector, a nucleic acid of interest is inserted into the viral genome in the place of certain viral sequences to produce a virus that is replication-defective. In order to produce virions, a packaging cell line containing the gag, pol, and env genes but without the LTR and psi components is constructed (Mann et al. (1983) Cell 33:153). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas and Rubenstein (1988) “Retroviral Vectors”, In: Rodriguez and Denhardt ed. Vectors: A Survey of Molecular Cloning Vectors and their Uses. Stoneham:Butterworth; Temin, (1986) “Retrovirus Vectors for Gene Transfer: Efficient Integration into and Expression of Exogenous DNA in Vertebrate Cell Genome”, In: Kucherlapati ed. Gene Transfer. New York: Plenum Press; Mann et al., 1983, supra). The media containing the recombinant retroviruses is then collected, optionally concentrated, and used for gene transfer. Retroviral vectors are able to infect a broad variety of cell types. However, integration and stable expression require the division of host cells (Paskind et al. (1975) Virology 67:242).

A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a fusion protein of the present invention, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. A preferred retroviral vector is a pSR MSVtkNeo (Muller et al. (1991) Mol. Cell Biol. 11:1785 and pSR MSV(Xbal) (Sawyers et al. (1995) J. Exp. Med. 181:307) and derivatives thereof. For example, the unique BamHI sites in both of these vectors can be removed by digesting the vectors with BamHI, filling in with Klenow and religating to produce pSMTN2 and pSMTX2, respectively, as described in PCT/US96/09948 by Clackson et al. Examples of suitable packaging virus lines for preparing both ecotropic and amphotropic retroviral systems include Crip, Cre, 2 and Am.

Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11 524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Other Viral Systems

Other viral vector systems that may have application in gene therapy have been derived from herpes virus, e.g., Herpes Simplex Virus (U.S. Pat. No. 5,631,236 by Woo et al., issued May 20, 1997), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth,; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxvirus, an arena virus, a vaccinia virus, a polio virus, and the like. In particular, herpes virus vectors may provide a unique strategy for persistence of the recombinant gene in cells of the central nervous system and ocular tissue (Pepose et al., (1994) Invest Ophthalmol Vis Sci 35:2662-2666). They offer several attractive features for various mammalian cells (Friedmann (1989) Science, 244:1275-1281; Ridgeway, 1988, supra; Baichwal and Sugden, 1986, supra; Coupar et al., 1988; Horwich et al.(1990) J.Virol., 64:642-650).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al,, 1990, supra). This suggested that large portions of the genome could be replaced with foreign genetic material. The hepatotropism and persistence (integration) were particularly attractive properties for liver-directed gene transfer. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al. (1991) Hepatology, 14:124A).

Administration of Viral Vectors

Generally the viral particles are transferred to a biologically compatible solution or pharmaceutically acceptable delivery vehicle, such as sterile saline, or other aqueous or non-aqueous isotonic sterile injection solutions or suspensions, numerous examples of which are well known in the art, including Ringer's, phosphate buffered saline, or other similar vehicles. Delivery of the recombinant viral vector can be carried out via any of several routes of administration, including intramuscular injection, intravenous administration, subcutaneous injection, intrahepatic administration, catheterization (including cardiac catheterization), intracranial injection, nebulization/inhalation or by instillation via bronchoscopy.

Preferably, the DNA or recombinant virus is administered in sufficient amounts to transfect cells within the recipient's target cells, including without limitation, muscle cells, liver cells, various airway epithelial cells and smooth muscle cells, neurons, cardiac muscle cells, etc. and provide sufficient levels of transgene expression to provide for observable ligand-responsive secretion of a target protein, preferably at a level providing therapeutic benefit without undue adverse effects.

Optimal dosages of DNA or virus depends on a variety of factors, as discussed previously, and may thus vary somewhat from patient to patient. Again, therapeutically effective doses of viruses are considered to be in the range of about 20 to about 50 ml of saline solution containing concentrations of from about 1×10⁷ to about 1×10¹⁰ pfu of virus/ml, e.g. from 1×108 to 1×10⁹ pfu of virus/ml.

Uses:

1. Constitutive High-level Expression in Gene Therapy

Gene therapy often requires controlled high-level expression of a therapeutic gene. By supplying the transgene under the control of the RSV promoter in accordance with the methods of this invention, considerably higher levels of gene expression can be obtained relative to natural promoters or enhancers. Thus, one application of this invention to gene therapy is the delivery to a primate of a desired therapeutic gene operably linked to an RSV promoter.

This method may be employed to increase the efficacy of many gene therapy strategies by substantially elevating the expression of an exogenous therapeutic gene, allowing expression to reach therapeutically effective levels. Examples of therapeutic genes that would benefit from this strategy are genes that encode secreted therapeutic proteins, such as cytokines (e.g., IL-2, IL-4, IL-12), CFTR (see e.g. Grubb et al, 1994, Nature 371:802-6), growth factors (e.g., VEGF), antibodies, caogulation factors such as Factor VIII:c and Factor IX, and soluble receptors. Other candidate therapeutic genes are disclosed in PCT/US93/01617. This strategy may also be used to increase the efficacy of “intracellular immunization” agents, molecules like ribozymes, antisense RNA, and dominant-negative proteins, that act either stoichiometrically or by competition. Examples include agents that block infection by or production of HIV or hepatitis virus and agents that antagonize the production of oncogenic proteins in tumors.

The method may also be employed to introduce the RSV promoter into the chromosome by homologous recombination, thereby placing an endogenous gene under the control of this strong promoter.

2. Regulated Gene Therapy

The efficacy of gene therapy may be enhanced in some cases through regulated, rather than constitutive, expression. For example, secretion of erythropoietin is normally regulated, and constant high level expression of the protein can even be toxic. Several regulated expression systems have been developed to deal with this problem, each of which can be used with the methods of this invention.

A. Dimerization-based Systems

In certain embodiments, two fusion proteins are encoded by the transgene, one or both of which is under the control of the RSV promoter. The first fusion protein contains a ligand binding domain linked to a transcription activation domain; the second fusion protein contains a ligand binding domain linked to a DNA binding domain. The target gene to be expressed is operatively linked to an expression control sequence to which the DNA binding domain binds. In this case, the ligand is at least divalent and functions as a dimerizing agent by binding to the two fusion proteins and forming a cross-linked heterodimeric complex which activates target target gene expression. See e.g. WO 94/18317, WO 96/20951, WO 96/06097, WO 97/31898, WO 96/41 865, and PCT US98/1 7723, the contents of which are incorporated herein by reference.

In the cross-linking-based dimerization systems the fusion proteins can contain one or more ligand binding domains (in some cases containing two, three or four such domains) and can further contain one or more additional domains, heterologous thereto, including e.g. a DNA binding domain, transcription activation domain, etc.

In general, any ligand/ligand binding domain pair may be used in such systems. For example, ligand binding domains may be derived from an immunophilin such as an FKBP, cyclophilin, FRB domain, hormone receptor protein, antibody, etc., so long as a ligand is known or can be identified for the ligand binding domain.

For the most part, the receptor domains will be at least about 50 amino acids, and fewer than about 350 amino acids, usually fewer than 200 amino acids, either as the natural domain or truncated active portion thereof. Preferably the binding domain will be small (<25 kDa, to allow efficient transfection in viral vectors), monomeric, nonimmunogenic, and should have synthetically accessible, cell permeant, nontoxic ligands as described above.

Preferably the ligand binding domain is for (i.e., binds to) a ligand which is not itself a gene product (i.e., is not a protein), has a molecular weight of less than about 5 kD and preferably less than about 3 kD, and is cell permeant. In many cases it will be preferred that the ligand does not have an intrinsic pharmacologic activity or toxicity which interferes with its use as a transcription regulator.

The DNA sequence encoding the ligand binding domain can be subjected to mutagenesis for a variety of reasons. The mutagenized ligand binding domain can provide for higher binding affinity, allow for discrimination by a ligand between the mutant and naturally occurring forms of the ligand binding domain, provide opportunities to design ligand-ligand binding domain pairs, or the like. The change in the ligand binding domain can involve directed changes in amino acids known to be involved in ligand binding or with ligand-dependent conformational changes. Alternatively, one may employ random mutagenesis using combinatorial techniques. In either event, the mutant ligand binding domain can be expressed in an appropriate prokaryotic or eukaryotic host and then screened for desired ligand binding or conformational properties. Examples involving FKBP, cyclophilin and FRB domains are disclosed in detail in WO 94/18317, WO 96/06097, WO 97/31898 and WO 96/41865). Illustrative of this situation is to modify FKBP12's Phe36 to Ala and/or Asp37 to Gly or Ala to accommodate a substituent at positions 9 or 10 of FK506 or FK520. In particular, mutant FKBP12 moieties which contain Val, Ala, Gly, Met or other small amino acids in place of one or more of Tyr26, Phe36, Asp37, Tyr82 and Phe99 are of particular interest as receptor domains for FK506-type and FK-520-type ligands containing modifications at C9 and/or C10. Illustrative mutations of current interest in FKBP domains also include the following:

TABLE 1
Entries identify the native amino acid by single letter code and sequence
position, followed by the replacement amino acid in the mutant. Thus,
F36V designates a human FKBP12 sequence in which phenylalanine at
position 36 is replaced by valine. F36V/F99A indicates a double mutation
in which phenylalanine at positions 36 and 99 are replaced by valine
and alanine, respectively.

| | | | |
|---|---|---|---|
| F36A | Y26V | F46A | W59A |
| F36V | Y26S | F48H | H87W |
| F36M | D37A | F48L | H87R |
| F36S | I90A | F48A | F36V/F99A |
| F99A | I91A | E54A/F36V/F99G | F99G |
| F46H | E54K/F36M/F99A | Y26A | F46L |
| V55A | F36M/F99G | | |

Illustrative examples of rapamycin-binding domains are those which include an approximately 89-amino acid rapamycin-binding domain from FRAP, e.g., containing residues 2025-211 3 of human FRAP. Another preferred portion of FRAP is a 93 amino acid fragment consisting of amino acids 2021-2113. Similar considerations apply to the generation of mutant FRAP-derived domains which bind preferentially to rapamycin analogs (rapalogs) containing modifications (i.e., are ‘bumped’) relative to rapamycin in the FRAP-binding effector domain. For example, one may obtain preferential binding using rapalogs bearing substituents other than -OMe at the C7 position with FRBs based on the human FRAP FRB peptide sequence but bearing amino acid substitutions for one of more of the residues Tyr2038, Phe2039, Thr2098, Gln2099, Trp2101 and Asp2102. Exemplary mutations include Y2038H, Y2038L, Y2038V, Y2038A, F2039H, F2039L, F2039A, F2039V, D2102A, T2098A, T2098N, T2098L, and T2098S. Rapalogs bearing substituents other than —OH at C28 and/or substituents other than ═O at C30 may be used to obtain preferential binding to FRAP proteins bearing an amino acid substitution for Glu2032. Exemplary mutations include E2032A and E2032S. Proteins comprising an FRB containing one or more amino acid replacements at the foregoing positions, libraries of proteins or peptides randomized at those positions (i.e., containing various substituted amino acids at those residues), libraries randomizing the entire protein domain, or combinations of these sets of mutants are made using the procedures described above to identify mutant FRAPs that bind preferentially to bumped rapalogs. See, for example, U.S. Ser. No. 09/012,097, the contents of which are incorporated herein by reference.

Other macrolide binding domains useful in the present invention, including mutants thereof, are described in the art. See, for example, WO96/41865, WO96/1 3613, WO96/061 1 1, WO96/061 10, WO96/06097, WO96/12796, WO95/05389, WO95/02684, WO94/18317, each of which is expressly incorporated by reference herein.

The ability to employ in vitro mutagenesis or combinatorial modifications of sequences encoding proteins allows for the production of libraries of proteins which can be screened for binding affinity for different ligands. For example, one can totally randomize a sequence of 1 to 5, 10 or more codons, at one or more sites in a DNA sequence encoding a binding protein, make an expression construct and introduce the expression construct into a unicellular microorganism, and develop a library. One can then screen the library for binding affinity to one or desirably a plurality of ligands. The best affinity sequences which are compatible with the cells into which they would be introduced can then be used as the ligand binding domain. The ligand would be screened with the host cells to be used to determine the level of binding of the ligand to endogenous proteins. A binding profile could be defined weighting the ratio of binding affinity to the mutagenized binding domain with the binding affinity to endogenous proteins. Those ligands which have the best binding profile could then be used as the ligand. Phage display techniques, as a non-limiting example, can be used in carrying out the foregoing.

In other embodiments, antibody subunits, e.g. heavy or light chain, particularly fragments, more particularly all or part of the variable region, or fusions of heavy and light chain to create single chain antibodies, can be used as the ligand binding domain. Antibodies can be prepared against haptenic molecules which are physiologically acceptable and the individual antibody subunits screened for binding affinity. The cDNA encoding the subunits can be isolated and modified by deletion of the constant region, portions of the variable region, mutagenesis of the variable region, or the like, to obtain a binding protein domain that has the appropriate affinity for the ligand. In this way, almost any physiologically acceptable haptenic compound can be employed as the ligand or to provide an epitope for the ligand. Instead of antibody units, natural receptors can be employed, where the binding domain is known and there is a useful ligand for binding.

In yet another embodiment of the invention, the DNA binding unit is linked to more than one ligand binding domain. For example, a DNA binding domain can be linked to at least 2, 3, 4, or 5 ligand binding domains. A DNA binding domain can also be linked to at least 5 ligand binding domains or any number of ligand binding domains, In such embodiments, the ligand binding domains can be, by illustration, linked to each other in a linear array, by linking the NH2-terminus of one ligand binding domain to the COOH-terminus of another ligand binding domain. Thus, more than one molecule of a chimeric transcription factor can be cross-linked to a single DNA binding domain in the presence of a divalent ligand.

B. Allostery-based systems

In other embodiments, the ligand binding event is thought to result in an allosteric change in the chimeric transcription regulatory protein leading to binding of the fusion protein to a target DNA sequence [see e.g. U.S. Ser. Nos. 5,654,168 and 5,650,298 (tet systems), and WO 93/23431 and WO 98/18925 (RU486-based systems)] or to another protein [see e.g. WO 96/37609 and WO 97/38117 (ecdysone/RXR-based systems)], in either case, modulating target gene expression.

The methods of the present invention are also useful in such ligand-dependent transcription regulation switches based on allosteric changes in a chimeric transcription regulatory protein. In such cases, the expression of the chimeric transcription regulatory protein is controlled by the RSV promoter. One such switch employs a deletion mutant of the human progesterone receptor which no longer binds progesterone or any known endogenous steroid but can be activated by the orally active progesterone antagonist RU486, described, e.g, in Wang et al. (1994) Proc. Natl. Acad. Sci. U.S.A. 91:8180. The transcription factor in this system generally consists of a ligand binding domain for binding RU486, a DNA binding domain such as GAL4 and an activation domain, typically VP16. Activation was demonstrated, e.g, in cells transplanted into mice using doses of RU486 (5-50 mg/kg) considerably below the usual dose for inducing abortion in humans (10 mg/kg). However, according to the art describing this system, the induction ratio in culture and in animals was rather low. Thus, transcription would be controlled in primates according to the methods of this invention, by expressing in a primate a chimeric transcription regulatory protein comprising a ligand binding domain for binding RU486, a DNA binding domain and a transcription activation domain under the control of the RSV promoter. Upon administration of RU486 to the primate, expression of a target gene responsive to the presence of the ligand would be activated.

Another such system is referred to as the ecdysone inducible system. Early work demonstrated that fusing the Drosophila steroid ecdysone (Ec) receptor (EcR) Ec-binding domain to heterologous DNA binding and activation domains, such as E. coli lexA and herpesvirus VP16 permits ecdysone-dependent activation of target genes downstream of appropriate binding sites (Christopherson et al. (1992) Proc. Natl. Acad. Sci. U.S.A. 89:6314). An improved ecdysone regulation system has been reported, using the DNA binding domain of the EcR itself. In this system, the chimeric transcription regulatory protein is provided as two proteins: (1) a truncated, mutant EcR fused to herpes VP16 and (2) the mammalian homolog (RXR) of Ultraspiracle protein (USP), which heterodimerizes with the EcR (No et al. (1996) Proc. Natl. Acad. Sci. U.S.A. 93:3346). In this system, because the DNA binding domain was also recognized by a human receptor (the human farnesoid X receptor), it was altered to a site recognized only by the mutant EcR. Thus, the invention provides an ecdysone inducible system, in which a truncated mutant EcR is fused to at least one subunit of a transcription activator of the invention, expressed under the control of an RSV promoter. The chimeric transcription regulatory protein further comprises USP, thereby providing high level induction of transcription of a target gene having the EcR target sequence, dependent on the presence of ecdysone.

In another embodiment, the inducible system comprises the E. coli tet repressor (TetR), which binds to tet operator (tetO) sequences upstream of target genes. In the presence of tetracycline, or an analog, which bind to tetR, DNA binding is abolished and thus transactivation is abolished. This system, in which the TetR had previously been linked to transcription activation domains, e.g, from VP16, is generally referred to as an allosteric “off-switch” described by Gossen and Bujard (Proc. Natl. Acad. Sci. U.S.A. (1992) 89:5547) and in U.S. Pat. Nos. 5,464,758; 5,650,298; and 5,589,362 by Bujard et al. Furthermore, depending on the concentration of the antibiotic in the culture medium (0-1 mu g/ml), target gene expression can be regulated over concentrations up to several orders of magnitude. Thus, the system reportedly not only allows differential control of the activity of an individual gene in eukaryotic cells but also is suitable for creation of “on/off” situations for such genes in a reversible way. This system provides target gene expression in the absence of tetracycline or an analog. Thus, the invention described herein provides for expression of the tetracycline-responsive fusion protein under the control of the RSV promoter.

In another embodiment, a “reverse” Tet system is used, again based on a DNA binding domain that is a mutant of the E. coli TetR, but which binds to TetO in the presence of Tet. As described above for the RU486-based system, the methods of this invention would be used to control expression of a target gene in primates, by expressing in the primate a fusion protein comprising a ligand binding domain for binding tetracycline or an analog thereof, a DNA binding domain and a transcription activation domain under the control of the RSV promoter. Administration of a ligand that binds the ligand binding domain of the fusion protein would activate expression of a target gene responsive to said ligand.

A tetR domain useful in the practice of this invention may comprise a naturally occurring peptide sequence of a tetR of any of the various classes (e.g. class A, B, C, D or E) (in which case the absence of the ligand stimulates target gene transcription), or more preferably, comprises a mutated tetR which is derived from a naturally occurring sequence from which it differs by at least one amino acid substitution, addition or deletion. Of particular interest are those mutated tetR domains in which the presence of the ligand stimulates binding to the TetO sequence, usually to induce target gene transcription in a cell engineered in accordance with this invention. For example, mutated tetR domains include mutated Tn10-derived tetR domains having an amino acid substitution at one or more of amino acid positions 71, 95, 101 and 102. By way of further illustration, one mutated tetR comprises amino acids 1-207 of the Tn10 tetR in which glutamic acid 71 is changed to lysine, aspartic acid 95 is changed to asparagine, leucine 101 is changed to serine and glycine 102 is changed to aspartic acid. Ligands include tetracycline and a wide variety of analogs and mimics of tetracycline, including for example, anhydrotetracycline and doxycycline. Target gene constructs in these embodiments contain a target gene operably linked to an expression control sequence including one or more copies of a DNA sequence recognized by the tetR of interest, including for example, an upstream activator sequence for the appropriate tet operator. See e.g. U.S. Pat. No. 5,654,168. Additional information on mutated tetR-based systems is provided above and in patent documents cited previously.

The full contents of all references cited in this document, including references from the scientific literature, issued patents and published patent applications, are hereby expressly incorporated by reference.

The following examples contain important additional information, exemplification and guidance which can be adapted to the practice of this invention in its various embodiments and the equivalents thereof. The examples are offered by way of illustration only and should not be construed as limiting in any way. As noted throughout this document, the invention is broadly applicable and permits a wide range of design choices by the practitioner.

The practice of this invention will employ, unless otherwise indicated, conventional techniques of cell biology, cell culture, molecular biology, transgenic biology, microbiology, recombinant DNA, immunology, virology, pharmacology, chemistry, and pharmaceutical formulation and administration which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### Contructs Encoding Transgenes Operably Linked to an RSV Promoter

Cloning of RSV Enhancer:

The RSV enhancer was obtained from pREP8 (Invitrogen) as a 677 bp Sall-BamHI fragment and subcloned into pBS/SK+ (Stratagene) to generate pBS-RSV. pBS-RSVm4 was created by mutagenizing pBS-RSV with the following four oligonucleotides to create appropriate flanking restriction enzyme sites and to eliminate undesired internal restriction enzyme sites:

VR195: Add BgIII site at 3′ end

GCTAGCAAGCTTGagatctGCCGCTCGAGGC

VR196: Knockout internal EcoRI site

GGACGAACCACTaAATTCCGCATTGC

VR197: Knockout internal Mlul site

CGGGCCAGATATtCGCGTATCTGAG

VR198: Add Mlul site at 5′ end

cgagGTCGACCacgcgtCATGTTTGACAG

The resulting sequence containing the RSV enhancer/promoter as an Mlul-BgIII fragment is:

acgcgtcatgtttgacagcttatcatcgcagatccgtatggtgcactctcagtacaatctgctctgatgccgcatagtta
agccagtatctgctccctgcttgtgtgttggaggtcgctgagtagtgcgcgagcaaaatttaagctacaacaaggcaagg
cttgaccgacaattgcatgaagaatctgcttagggttaggcgttttgcgctgcttcgcgatgtacgggccagatattcgc
gtatctgaggggactagggtgtgtttaggcgaaaagcggggcttcggttgtacgcggttaggagtcccctcaggatatag
tagtttcgcttttgcatagggagggggaaatgtagtcttatgcaatactcttgtagtcttgcaacatggtaacgatgagt
tagcaacatgccttacaaggagagaaaaagcaccgtgcatgccgattggtggaagtaaggtggtacgatcgtgccttatt
aggaaggcaacagacgggtctgacatggattggacgaaccactaaattccgcattgcagagatattgtatttaagtgcct
agctcgatacaataaacgccatttgaccattcaccacattggtgtgcacctccaagctgggtaccagctgctagcaagct
tgagatct

Generation of pZA.RSV.rhEpo

The gene for rhesus erythropoietin was cloned from rhesus kidney and subcloned into a vector containing the CMV promoter, chimeric intron and poly A sequence as described in Ye et al., Science 283:88-91 (1999). To generate the RSV containing plasmid, the RSV promoter was subcloned from pAAV-RSV-TF1 Nc (described below.)

Generation of pAAV-RSV-TF1 Nc.

Here we describe the construction of an adeno-associated virus (pAAV-RSV-TF1 Nc) containing a bicistronic sequence encoding a first chimeric protein having a nuclear localization signal (NLS) from c-myc fused to a ligand-binding domain (FRB-T2098L) and a transcriptional activation domain (from p65) and a second chimeric protein having an NLS from c-myc fused to a ligand-binding domain (copies of FKBP) and a DNA-binding domain (ZFHD1). The two cistrons are separated by an internal ribosome entry sequence (IRES). Expression of the chimeric proteins is under control of an RSV enhancer. A human growth hormone (hGH) 3′ UTR, containing a polyA sequence, is located downstream of the bicistronic region.

To generate a chimeric protein containing the NLS from c-myc (N₂) fused to FRB-T2098L (R_{H1}) and p65, an Xbal-BamHI fragment from CGNN-R_{H1}p65 (more fully described in U.S. Ser. No. 09/076,369, which is incorporated herein by reference) was cloned into pC₅EN to yield pC₅EN-R_{H1}p65. To replace the HA epitope tag and SV40 NLS, the following two oligonucleotides were annealed and cloned between the EcoRI-Xbal sites of pC₅EN-R_{H1}p65 to yield pC₅N₂-R_{H1}S:

VR204: aattccagaagccaccATGGACTATCCTGCTGCCAAGAGGGTCAAGTTGGACT

VR205: CTAGAGTCCAACTTGACCCTCTTGGCAGCAGGATAGTCCATggtggcttctgg

To generate a chimeric protein containing the NLS from c-myc fused to 3 copies of FKBP and the ZFHD1 DNA-binding domain, an Xbal-BamHI fragment from CGNN-ZFHD1-3xFKBP (U.S. Ser. No. 09/076,369) was cloned into PC5N₂-RH1S to yield pC₅N₂-Z1 F3.

An Ncol-BamHI fragment containing N₂-Z1F3 was then cloned into pBS-IRES to place the c-myc NLS-ZFHD1-3xFKBP fusion protein downstream of the IRES from encephalomyocarditis virus (pBS-IRES-N₂-Z1F3).

The bicistronic contruct pC₅N₂-R_{H1}S/Z1F3 was then generated by cloning a BgIII-BamHI fragment from pBS-IRES-N₂-Z1F3 into the BamHI site of pC₅N₂-R_{H1}S.

pAAV-PL1-H3S is a derivative of pSub2Ol (Samulski et al. (1987) J. Virol. 61:3096) in which an Xbal fragment between the AAV ITRs (containing the rep and cap genes of the virus) was replaced with a polylinker and stuffer sequence. An Mlul-Xhol fragment from pC₅N₂-R_{H1}S/Z1 F3 was cloned into pAAV-PL1-H3S to create pAAV-CMV-TF1 N.

A 243 bp BamHI-Xhol fragment containing the 3'UTR from hGH was obtained by PCR from the plasmid pOGH (Selden et al. (1986) Mol. Cell.Biol. 6:3173) using the following oligonucleotides:

VR 149: CGGCGGATCCtgcccgggtggcatccctg

VR150: gccgCTCGAGgatcGGCGCGCCcagcttggttcccgaatag

The 3′ UTR of pAAV-CMV-TF1N was replaced with that of hGH by insertion of the BamHI-Xhol fragment to create pAAV-CMV-TF1Nc.

Finally, the CMV enhancer of pAAV-CMV-TF1Nc was replaced by that of RSV by insertion of an Mlul-BgIII fragment from pBS-RSVm4 to create pAAV-RSV-TF1N.

Cloning RSV Upstream of the Gal4-DBD/hPR-LBD/p65-AD Fusion Gene

The plasmid pSwitch (Invitrogen) expresses the Gal4-DBD/hPR-LBD/p65-AD fusion gene under control of a promoter containing four Gal4 concensus binding sites and a Herpes Simplex Virus thymidine kinase minimal promoter (Gal4/HSV TK promoter).

To put expression of the Gal4-DBD/hPR-LBD/p65-AD fusion gene under control of the RSV enhancer the plasmid pSwitch (Invitrogen) is first mutagenized with the following two oligonucleotides to insert an Mlul site upstream and a Spel site downstream of the Gal4/HSV TK promoter to create pSwitch/Mlul/Spel.

VR1000: gcttcgacctgcaCgcGtgcaagctcgaatg

VR1001: cccggtgtcttctACTAGTgtcaaaacagcgtgg

The Gal4/HSV TK promoter is then replaced by the RSV enhancer by inserting an Mlul-Spel fragment from pBS-RSVm4 into pSwitch/Mlul/Spel to create pRSV-Switch.

Cloning RSV Upstream of the Tet-off and Tet-on Transcription Factors

The plasmids pTet-Off and pTet-On (Clontech) express the tetracycline (tTA) and reverse tetracycline (rtTA) controlled transactivators fused to the VP16 activation domain from herpes simplex virus from the CMV enhancer. To put expression of the tTA-VP16 and rtTA-VP16 transactivators under control of the RSV enhancer the plasmids pTet-Off and pTet-On are first mutagenized with the following oligonucleotides to insert an Ascl site upstream and a Spel site downstream of the CMV promoter to create pTet-Off/Ascl/Spel and pTet-On/Ascl/Spel:

VR1002: ctcatgtccaacaGGCGcgccatgttgaca

VR1003: ggtctatataagcagaActAgtttagtgaaccgtc

The CMV promoter is then replaced by the RSV enhancer by inserting an Mlul-Spel fragment from pBS-RSVm4 into pTet-Off/Ascl/Spel and pTet-On/Ascl/Spel to create pRSV-Tet-Off and pRSV-Tet-On.

### Example 2

### Long Term Effects of H2.rAAV.CMVrhEPO Delivered by Intramuscular Injection into Non-human Primates: Gene Expression, Clinical, Pathologic and Immunologic Effects

Recombinant adeno-associated virus vectors have been demonstrated to be good candidates for somatic gene transfer to striated muscle in murine studies. The purpose of the present study is to assess the efficacy and safety of H2.rAAV.CMVrhEPO. Presently, there is limited information on the ability of adeno-associated virus vectors for sustained and stable gene transfer in skeletal muscle in non-human primates, a model that more closely resembles humans than that of experimental rodent systems. The reporter molecule, rhEPO, is readily measurable in serum and provides a surrogate method for somatic gene transfer and expression in the target tissue. Measurement of the hematocrit will also be used as a downstream marker of the expression of the erythropoietin transgene. In addition to the primary objective of stable gene transfer and expression, clinical pathology and immunology measures for assessing safety will be performed. At the termination of the experiment, a necropsy will be performed with a gross pathological examination followed by a histopathological exam in selected tissues.

### Experimental Design:

1. Two non human primates (rhesus monkeys RQ1582 and 93B644) were randomly assigned to the study following determination of neutralizing antibody levels to adenovirus and adeno-associated virus as well as other baseline values. On Day 1 of the study, the animals were sedated and weighed. Blood draws for baseline clinical pathology studies, hematocrit (HCT), and EPO expression were taken. A pre-vector chest x-ray was also be performed.

2. Recombinant virus for these animals was produced in the Human Applications Laboratory (HAL) of the Institute for Human Gene Therapy, University of Pennsylvania. The virus used in this study as designated by the HAL Label is H₂rAAV/CMV-rmEpo, which is in fact the vector H2.rAAV.CMVrhEPO.

Two lots of virus were combined for this study: Lots 2 and 3. They are designated as follows:

H₂rAAV/CMV-rmEpo, HAL 12-17-97 L2, 2.29×10¹³ genomes/ml

H₂rAAV/CMV-rmEpo, HAL 12-23-97 L3, 7.90×10¹² genomes/ml

Viruses were stored in 10% glycerol/PBS at −65 to -80° C., and expired 6 months from date of preparation. All virus preparation was done under sterile conditions with sterile reagents by Human Applications Laboratory personnel.

H2.rAAV.CMVrhEPO was Intramuscularly injected into the vasta lateralis muscles. To identify the injection sites, the overlying skin at 10 sites was shaved and indelibly marked on day −1, one day prior to administration of virus suspension. On day 1, 1.0 ml of the vector suspension was injected with a 26 gauge needle at a tattooed skin site, through the fascia, and into the muscle. Prior to injection the syringe plunger was gently withdrawn and observed for any blood. A total of 5 injections per each quadriceps with a total volume of 10 ml was administered. For monkey RQ1582, this corresponded to 0.5×10¹³ genomes, and for monkey 93B644, this corresponded to 1×10¹³ genomes.

3. After vector administration, the animal was monitored daily for general observations. On select days listed below, the animal was sedated and the following parameters monitored: gene expression, hematocrit levels, clinical pathology, immunology, chest radiographs, and body weights and temperatures.

a. Gene Expression

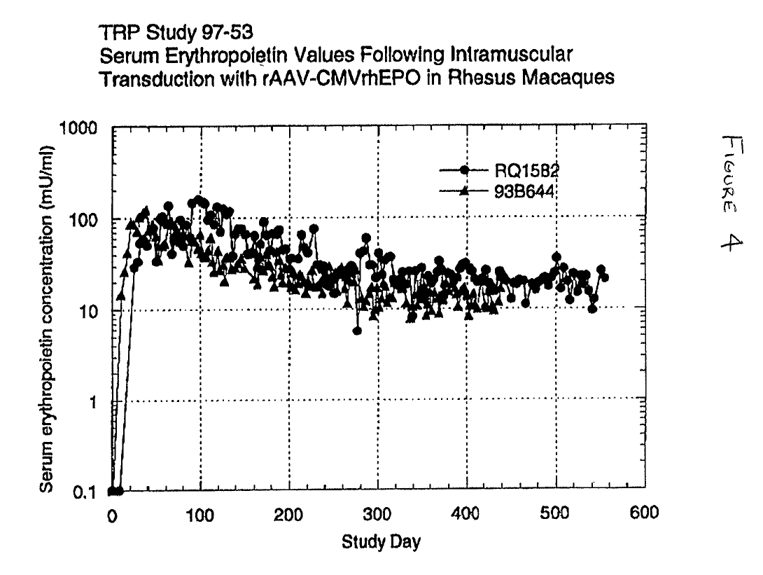

EPO levels were quantified using the Quantikine IVD human EPO, ELISA Kit, from R&D Systems Catalog # DEP00. The assay using 14 wells for standards and each animal sample is run using two different dilutions. The ability of the vector to express the transgene was monitored by an ELISA on serum samples from the animal. For the two weeks following the test article administration, the EPO levels were monitored weekly. They were then monitored twice a week for 5 weeks to determine a peak, then monitored weekly for the following month, and finally, every other week for the duration of the study. Blood samples for EPO expression were taken in a red top tube and the serum separated via centrifugation. These samples will be drawn on Study Days: 8, 15, 18, 22, 25, 29, 32, 36, 39, 43, 46, 50, 57, 64, 71, 78, 85, 99, 113, 127, 141, 155, 169, and 180. FIG. 4 shows the result of such an experiment, indicating that a peak serum EPO concentration of 100 mU/mi is reached by day 25.

b. Hematocrit

The expression of the transgene occasionally results in a change in the hematocrit of the animal which poses a threat to its general health. In an effort to monitor this potential problem, the hematocrit of the animal was measured on a regular basis. Initially, hematocrits were determined on a weekly basis for the first two weeks following test article administration, then increased to twice weekly for three months, then weekly for the duration of the study. The specific study days of HCT monitoring are: 8, 15, 18, 22, 25, 29, 32, 36, 39, 43, 46, 50, 53, 57, 60, 64, 67, 71, 74, 78, 81, 85, 88, 92, 95, 99, 102, 106, 109, 113, 120, 127, 134, 141, 148, 155,162, 169,176, 180.

The frequency for the determination of the hematocrit was to assess transduction efficacy and the possible requirements of therapeutic phlebotomy. When the hematocrit ≧65%, the veterinarian was notified and the experimental animal was phlebotomized of 7.0 ml/kg of blood, approximately 10% of blood volume with monitoring of vital signs (heart rate, respiratory rate, capillary refilling) over a 20 minute time interval.

c. Clinical Patholoay

Changes in the blood chemistries and blood profiles of the animal were monitored by the contract facility LabCorp, Inc. The parameters monitored included the CBCs with Differentials, partial thromboplastin time (PTT), prothrombin time(PT) and a variety of chemistries including liver function tests and muscle function tests. These items were monitored on samples from the animal at specific time points. Following the test article administration, the clinical pathology was monitored every other week for the first three months of the study and then only monthly for the remainder of the study. The specific timepoints of clinical pathology analysis were Study Days 15, 29, 43, 57, 71, 85, 99, 127, 155 and 180.

d. Immunology

To monitor the Immunologic changes in the animal, blood draws occurred Study Days 15, 29, 57, 85, 127, 155, and 180. The Immunologic parameters of cytokine secretion and lymphoproliferation were monitored on Days 15, 57, and 180. Neutralizing antibody response to both vector and transgene and westerns were run on the samples from Days 29, 57, 85, 127, 155, 180. At the timepoints in which the animal had blood drawn for immunology, it was also monitored for HCT, EPO expression, and clinical pathology.

e. Chest Xrays:

Previous studies involving gene transfer to skeletal muscle tissue have monitored the animal health using chest x-rays. The days of monitoring for this study was day 1, 8, 43, 85, and 180. The animal is sedated for the procedure. The timepoints selected for the monitoring of this parameter are timepoints in which all other parameters (HCT, EPO, Immunology, din path, body weights) are being monitored.

f. Body Weights and Temperatures

At all timepoints selected for monitoring, the animal will be sedated then weighed and have its body temperature taken via rectal thermometers. Body temperature will be taken twice at each timepoint, at least 10 minutes apart. Body weight is determined prior to any blood samples being taken.

4. The study ended on Day 180 when the animal was sedated, weighed, had blood drawn for clinical pathology, immunology, gene expression, and hematocrit assays. It also underwent a chest x-ray prior to sacrifice. The animal was euthanized and a partial necropsy performed. Gross pathology was observed and selected tissues taken for histopathological examination.

### Example 3

### Gene Expression, Clinical, Pathologic and Immunologic Effects of the AAV Vector AAV2.RSVrhEPO

General Design Procedures

1. On Day 1, the animal was weighed. Blood draws for baseline clinical pathology studies, Immunology, hematocrit and EPO expression were taken.

2. The monkey was treated with AAV2.RSVrhEPO at a dose of 2×10¹³ g.c./kg injected intramuscularly into the right and left vasta lateralis muscle. The vector was injected with a 26 gauge needle. Prior to injection, the syringe plunger was gently withdrawn and observed for any blood to prevent inadvertent intravenous delivery. A total of 10 injections of 1 ml each per leg were given, for a total of 10 injection sites. The vector was administered once on test day 1.

3. After vector administration, the animal was monitored daily for general observations. On select days listed below, the animal will be monitored for: gene expression, clinical pathology, immunology, chest radiographs, body weights and temperatures.

a. Gene Expression

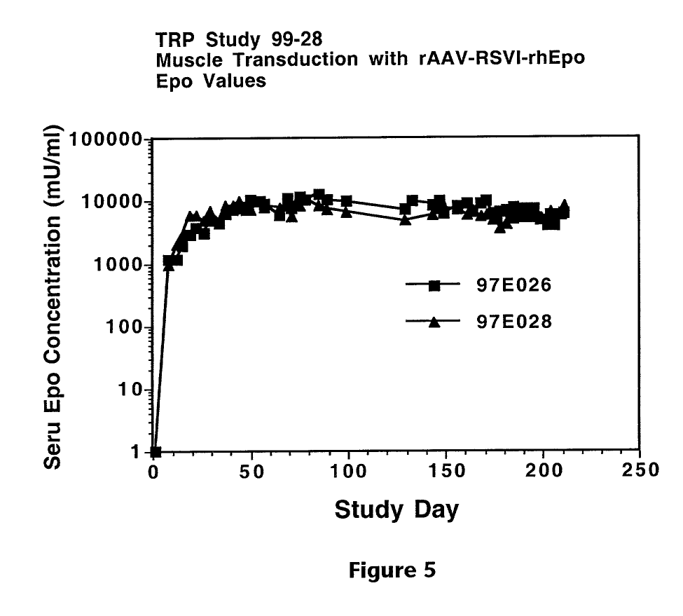

The ability of the vector to express the transgene is monitored for EPO expression by an ELISA on serum samples from the animals. Blood samples for EPO expression are taken in a red top tube whenever the hematocrits are evaluated and the serum separated via centrifugation. FIG. 5 shows the result of such an experiment, indicating that by day 60, the animal is expressing >1×10⁴ mU/ml of serum EPO.

b. Hematocrit

The expression of the transgene may result in a change in the hematocrits of the animals which poses a threat to their general health. In an effort to monitor this potential problem, the hematocrits of the animals are measured on a regular basis (see chart below). HCTs will be monitored on twice weekly status after the anticipated expression begins and will continue for the duration of the study. More frequent monitoring may be conducted if necessary due to high hematocrits, e.g. greater than 65%.

c. Clinical Pathology

Changes in the blood chemistries and blood profiles of the animals will be monitored by the contract facility LabCorp, Inc. These items will be monitored on samples from the animals at specific time points.

d. Immunology

The immunologic parameters of cytokine secretion and lymphoproliferation, neutralizing antibody response to both vector and transgene, and Westerns will be performed.

| Assay | Tube type |
|---|---|
| CTL, lympho, cytokines | Green top/heparin (GT) |
| NAB, Western | Red top serum separator (RT) |

e. Body Weights and Temperatures

At all timepoints selected for monitoring, the animal is sedated, then weighed and its body temperature is taken via rectal thermometers. Body weight is determined prior to any blood samples being taken.

4. The study was scheduled to end after approximately 6 months, however, the study has been continued since continued expression is observed. At the end of the in-life phase, the animals will be sedated, weighed, have blood drawn for clinical pathology, immunology, and gene expression. The animal will be euthanized and the necropsy performed. Gross pathology will be observed and select tissues taken for histopathological examination. Some tissues may be taken for analysis of the DNA from the AAV vectors for the presence of DNA or the integration of the AAV vector DNA. Gene transfer and transgene expression will be examined at injection sites and possibly at other sites using immunology or molecular biology techniques.

### Example 4

### ELISA for Measurement of Gene Expression

The assay kit used was the Quantikine IVD Human Erythropoietin ELISA Kit (R&D Systems, cat # DEP00), The procedure followed was essentially that of the manufacturer. Serum sample of at least 0.25 mls was collected from test animal. Serum not processed immediately was stored at −20° C.

Wash Buffer Concentrate was warmed to room temperature to remove any crystals that may have formed. 1× Wash Buffer was prepared by diluting 100 mL of Concentrate into 2.4 L of ddH₂O.

Substrate Solutions 1 and 2 were mixed together in equal volumes within 1 5 minutes of use. 200 ul of the resultant mixture is required per well. 100 uL of Epo Assay Diluent was pipetted into each well. 100 uL of Erythropoietin Standard, Erythropoietin Serum Control, or specimen was added per well. The plate was covered with the adhesive strip provided and incubated for 1 hour±5 minutes at room temperature on a horizontal orbital microplate shaker (0.12″ orbit) set at 500±100 rpm. Wells were washed 3 times with at least 400 ml 1× Wash Solution per wash. Samples and wash solution were removed from wells by flicking the plate and blotting on paper towels. Next, 200 ul of Epo Conjugate was added to each well and the plate was covered with a new adhesive strip. The plate was incubated for 1 hour±5 minutes at room temperature on a horizontal orbital microplate shaker set at 500±100 rpm.

The plate was inverted to remove liquid from wells, blotted on absorbent pad or paper towels and washed, repeating the process four times for a total of 5 washes. The wash was carried out by filling each well with 1× Wash Buffer (400 L) using a squirt bottle or multi-channel pipette. After the last wash, any remaining Wash Buffer was removed by decanting and blotting.

200 uL of freshly prepared Substrate Solution was added to each well and incubated for 20-25 minutes at room temperature on the bench top. Following the incubation, 100 uL of Stop Solution was added to each well and the optical density (O.D.) of each well was determined within 15 minutes, using a microplate reader set to 450 nm. If wavelength correction is available set to 600 nm. If wavelength correction is not available, subtract readings at 600 nm from the readings at 450 nm. This subtraction will correct for optical imperfections in the plate.

claims:

1. A method for genetically engineering a primate for expression of a desired gene, comprising introducing into the primate a transgene comprising an RSV promoter and a nucleic acid sequence heterologous to said RSV promoter.

2. The method of claim 1 wherein the transgene comprises an RSV promoter operably linked to a nucleic acid comprising a selected ORF.

3. The method of claim 1 wherein the transgene comprises an RSV promoter and primate nucleic acid sequence.

4. The method of any of claims 1-3 wherein the RSV promoter comprises a sequence selected from a long terminal repeat of a strain of the Rous Sarcoma Virus.

5. The method of claim 4 wherein the selected RSV promoter sequence contains at least 50 nucleotides.

6. The method of any of claims 1-3 wherein the RSV promoter comprises a sequence which hybridizes under stringent conditions to a sequence selected from a long terminal repeat of a strain of the Rous Sarcoma Virus.

7. The method of any of claims 1-3 wherein the RSV promoter comprises a sequence of at least 50 nucleotides present in nucleotides 90-612 of Seq ID #1.

8. The method of any of claims 1-3 wherein the RSV promoter comprises at least the sequence 550-612 of Seq ID #1.

9. The method of any of claims 1-3 wherein the RSV promoter comprises a sequence of at least 20 nucleotides present in nucleotides 90-612 of Seq ID #1, with up to 5 nucleic acid substitutions, insertions or deletions.

10. The method of any of claims 1-3 wherein the RSV promoter comprises the sequence 349-612 of Seq ID #1 or a nucleic acid which hybridizes thereto under stringent conditions.

11. The method of any of claims 1-3 wherein the RSV promoter comprises the sequence 126-612 of Seq ID #1 or a nucleic acid which hybridizes thereto under stringent conditions.

12. The method of any of claims 1-3 wherein the RSV promoter comprises the sequence 90-612 of Seq ID #1 or a nucleic acid which hybridizes thereto under stringent conditions.

13. The method of any of claims 1-12, wherein the transgene is packaged in a virus.

14. The method of claim 5, wherein the virus is selected from the group consisting of adenovirus, AAV, retrovirus, hybrid adeno-AAV, herpesvirus and lentivirus.

15. The method of any of claims 1-14, wherein the primate is a human.

16. The method of any of claims 1-15, wherein the transgene is introduced into the muscle of the primate.

17. The method of any of claims 1-15, wherein the transgene is introduced into the liver of the primate.

18. The method of any of claims 1-15, wherein the transgene is introduced into the central nervous system of the primate.

19. The method of any of claims 1-15, wherein the primate cells are engineered ex vivo and are introduced into the primate.

20. A method for genetically engineering a primate for regulatable expression of a target gene which method comprises introducing into the primate a transgene comprising an RSV promoter operably linked to at least one recombinant nucleic acid encoding one or more fusion proteins, wherein the one or more fusion proteins bind to a ligand and in the presence of said ligand modulate(s) the expression level of a target gene.

21. The method of claim 20 wherein the target gene is endogenous to the primate.

22. The method of claim 20 wherein the target gene is heterologous to the primate.

23. The method of claim 20 wherein the presence of the ligand increases the expression level of the target gene.

24. The method of claim 20 wherein the presence of the ligand decreases the expression level of the target gene.

25. The method of claim 20 wherein the fusion protein contains a ligand binding domain which is or is derived from an immunophilin, cyclophilin, FRB, antibiotic resistance or hormone receptor domain.

26. The method of claim 25 wherein the ligand binding domain is or is derived from FKBP, tetR, progesterone receptor or ecdysone receptor.

27. A primate cell containing and capable of expressing a transgene comprising an RSV promoter operably linked to at least one recombinant nucleic acid encoding one or more fusion proteins, wherein the one or more fusion proteins bind to a ligand and in the presence of said ligand modulate(s) the expression level of a target gene.

28. The cell of claim 27 wherein the target gene is endogenous to the primate.

29. The cell of claim 27 wherein the target gene is heterologous to the primate.

30. The cell of claim 27 wherein the presence of the ligand increases the expression level of the target gene.

31. The cell of claim 27 wherein the presence of the ligand decreases the expression level of the target gene.

32. The cell of claim 27 wherein the fusion protein contains a ligand binding domain which is or is derived from an immunophilin, cyclophilin, FRB, antibiotic resistance or hormone receptor domain.

33. The cell of claim 32 wherein the ligand binding domain is or is derived from FKBP, tetR, progesterone receptor or ecdysone receptor.
